(19) United States
(12) Patent Application Publication
(10) Pub. No.: US 2026/0038112 A1
(43) Pub. Date: Feb. 5, 2026

(54) BALANCE FUNCTION MANAGEMENT SYSTEM AND METHOD FOR GENERATING INFORMATION ON BALANCE FUNCTION STATUS AND PERFORMING BALANCE FUNCTION REHABILITATION PROGRAM BY TRACKING EYE AND HEAD POSITION CHANGES IN VIDEOS, RECORDING MEDIUM STORING PROGRAM FOR EXECUTING THE SAME, AND RECORDING MEDIUM STORING PROGRAM FOR EXECUTING THE SAME

(71) Applicants: NeuroEars Co., Ltd., Chuncheon-si (KR); Industry Academic Cooperation Foundation, Hallym University, Chuncheon-si (KR)

(72) Inventors: Sung Kwang HONG, Bundang-gu (KR); Eun-Cheon LIM, Gwanak-gu (KR); Ha Neul KWON, Anyang-si (KR); Dae Hee YOON, Gunpo-si (KR)

(21) Appl. No.: 18/911,361

(22) Filed: Oct. 10, 2024

(30) Foreign Application Priority Data
Aug. 5, 2024 (KR) 10-2024-0104114
Aug. 5, 2024 (KR) 10-2024-0104115

(51) Int. Cl. G06T 7/00; A61B 5/00; A61B 5/11; G06T 3/40; G06T 7/246; G06T 7/73

(52) U.S. Cl. CPC G06T 7/0012; A61B 5/0077; A61B 5/1128; A61B 5/4023; A61B 5/7264; A61B 5/7278; G06T 3/40; G06T 7/246; G06T 7/73; G06T 2207/10016; G06T 2207/20081; G06T 2207/20084; G06T 2207/30201

(57) ABSTRACT

A balance function management system includes at least one processor, and a memory that stores instructions executable by the processor and stores at least one artificial neural network model executed on a computing device, in which the at least one processor may input frame images of n videos of a subject captured by n (natural number) cameras to at least one artificial neural network model to acquire at least one of information related to head coordinates, coordinates of a pupil center, and eye phase changes of the subject according to an order of frame images of an m-th (natural number from 1 to n) video, and use the information to generate information on a balance function status or information related to head movement and eye movement for performing a balance function rehabilitation program.

### CROSS-REFERENCE TO RELATED APPLICATIONS

This application claims the priority of Korean Patent Applications No. 10-2024-0104114 and 10-2024-0104115 filed on Aug. 5, 2024, in the Korean Intellectual Property Office, the disclosures of which are incorporated herein by reference.

### BACKGROUND

### Field

The present disclosure relates to a balance function management system and method for generating information on a balance function status and a balance function rehabilitation program, and more particularly, to a balance function management system and method for generating information on a balance function status of a subject by tracking eye and head position changes of the subject in a video of the subject captured by at least one camera and performing a balance function rehabilitation program of the subject, a recording medium storing a program for executing the same, and a computer program stored in the recording medium.

### Description of the Related Art

The contents described in this section merely provide background information for an exemplary embodiment described in the present specification and do not necessarily constitute the related art.

The human body maintains balance by fixing a gaze regardless of head movement. An inner ear detects head movement and transmits the detected signal to a brain. When the signal detected by the inner ear is transmitted to the brain, the brain stimulates extraocular muscles of the eye to induce a vestibulo-ocular reflex that moves eyes in an opposite direction to the head movement, thereby contributing to maintaining the balance. Therefore, when the inner ear is damaged or there is a problem in the signal integration process that transmits head movement recognized by the inner ear to the brain, the vestibulo-ocular reflex is not induced, so the human body may not maintain balance and feel severe dizziness.

In clinical practice, it is determined whether the balance function is abnormal through a test that evaluates eye movement in comparison to the head movement. To this end, in the clinical practice, it is determined whether the balance function is abnormal by measuring the speed of the eye movement compared to that of the head movement using an inertial measurement unit (IMU) sensor (gyroscope, accelerometer, etc.) that measures the speed of the head movement and an eye-tracking video camera that captures the eye movement.

Meanwhile, the acute dizziness occurs initially when the abnormality in the balance function occurs, but the dizziness gradually improves over time due to the compensatory action of a vestibular system. The dizziness is improved due to the compensatory action of the vestibular system, and through the compensatory action, our body contributes to improving symptoms by activating the damaged balance function, i.e., proprioceptive senses such as eyes and muscles instead of the inner ear. The vestibular compensation accelerates as the eyes and head move more, and the vestibular rehabilitation treatment used in the clinical practice is used as the principle of activating the vestibular compensation by moving the eyes and head.

Examples of the existing vestibular rehabilitation methods include a method in which a therapist uses business cards, etc., to induce patient's gaze movement and move a patient's head, a method in which a virtual reality device, etc., is used recently to move patient's head and eyes, etc. However, the conventional rehabilitation exercise method using business cards, etc., does not guarantee accurate rehabilitation exercise for a patient. In addition, the rehabilitation exercise method using the virtual reality device may provide feedback to a patient by tracking the patient's head and eye movement, allowing more accurate rehabilitation exercise, but requires additional devices such as the virtual reality device, which has limitations in actual clinical practice.

Although a nystagmus test, which evaluates the vestibulo-ocular reflex when the dizziness occurs, has been proven to be cost-effective and superior in diagnostic sensitivity to MRI, it has the problem of difficulty in conducting the test and interpretation. Therefore, there is no device for conducting the nystagmus test. As a result, the nystagmus test is not well utilized actually in emergency rooms or primary hospitals where dizziness patients visit the most, and thus, the dizziness is often diagnosed by relying on unnecessary imaging tests or blood tests. This causes waste of medical expenses in terms of cost effectiveness, and often leads to poor prognosis for patients due to lack of proper diagnosis. In particular, it is difficult for patients to perform vestibular rehabilitation treatment on their own to recover from acute dizziness symptoms, which delays recovery from the dizziness and causes a decrease in quality of life.

### RELATED ART DOCUMENT

### Patent Document

Korean Patent Laid-Open Publication No. 10-2004-0107677 (Dec. 23, 2004)

### SUMMARY

An object to be achieved by the present specification is to provide a balance function management system for generating information on a balance function status and a balance function rehabilitation program.

The present specification is not limited to the above-described problems, and other problems that are not described may be obviously understood by those skilled in the art from the following description.

A balance function management system according to an exemplary embodiment of the present specification may include: at least one processor; and a memory that stores instructions executable by the processor and stores at least one artificial neural network model executed on a computing device, in which the at least one processor may input frame images of n videos of a subject captured by n (natural number) cameras to at least one artificial neural network model to acquire at least one of information related to head coordinates, coordinates of a pupil center, and eye phase changes of the subject according to an order of frame images of an m-th (natural number from 1 to n) video, and use the information to generate information related to head movement and eye movement for generating information on a balance function status or performing a balance function rehabilitation program.

The at least one processor may include: a head coordinate acquirer that executes a first artificial neural network model stored in the memory, and inputs frame images of the m-th video or multi-frame images concatenating frame images of n videos to artificial neural network model to acquire the the first information related to the head coordinates according to the m-th video; an eye coordinate acquirer that executes a second artificial neural network model stored in the memory, and inputs the information related to the head coordinates to the second artificial neural network model to acquire information related to coordinates of a pupil center according to the m-th video; and a phase change acquirer that executes a third artificial neural network model stored in the memory, and inputs information related to coordinates of a pupil center according to a time sequence of the multi-frame image or the frame image of the m-th video to the third artificial neural network model to acquire the information related to the eye phase changes according to the m-th video.

The first artificial neural network model may be an artificial neural network model trained by allowing the at least one processor to use, as training data, facial feature points extracted from frame images of at least one video in which a human face is captured or multi-frame images in which frame images of multiple videos in which a human face is captured are concatenated and coordinates of the feature points.

The feature point may be a feature point positioned within a preset area in the frame images or the multi-frame images.

The second artificial neural network model may be an artificial neural network model trained to generate information related to coordinates of a pupil center by allowing the at least one processor to use, as training data, eye area images extracted to include an eye from frame images of at least one video in which a human face is captured or multi-frame images in which frame images of multiple videos in which a human face is captured are concatenated.

The at least one processor may generate pupil area images in which an area occupied by the pupil and the remaining area have different pixel values in the eye area images, and train the second artificial neural network model using the pupil area images or an array of pixel values of the pupil area images as the training data.

The at least one processor may train the second artificial neural network model to generate eye feature points and coordinate information of the feature points from the eye area images, and to generate horizontal coordinate values and vertical coordinate values of the pupil center using coordinates of a plurality of preset feature points.

The memory may store data of at least one virtual object, and the second artificial neural network model may be an artificial neural network model trained to generate information related to coordinates of a pupil center by allowing the at least one processor to use training data that includes a parameter value that changes at least one of parameters related to head rotation, eye rotation, and camera settings of the virtual object and an image of the virtual object acquired according to the parameter value.

The third artificial neural network model may be an artificial neural network model trained to generate an eye rotation value by allowing the at least one processor to use, as training data, information related to eye phase changes generated according to a time sequence of an eye area image extracted to include an eye from frame images of at least one video in which a human face is captured or multi-frame images in which frame images of multiple videos in which a human face is captured are concatenated.

The third artificial neural network model may be an artificial neural network model trained by allowing the at least one processor to use information comparing pixel values of an area occupied by an iris between eye area images corresponding to each frame image of each video, or to each frame image of each video in the multi-frame images.

The at least one processor may calculate a size of an area occupied by a pupil in the eye area images and adjust a size of a target eye area image using a size of an area occupied by a pupil in a preceding eye area image.

The at least one processor may include: a head movement generator that generates information related to head movement in an m-th (natural number from 1 to n) video using the information related to the head coordinates generated from at least one artificial neural network model; an eye movement generator that generates information related to eye movement in the m-th video using information related to coordinates of a pupil center or eye phase changes generated from at least one artificial neural network model; a speed information generator that generates information related to head and eye movement speeds in the m-th video using the information related to the head movement and the eye movement; and a balance function status information generator that generates information on a balance function status of the subject using the information related to the head and eye movement speeds.

The speed information generator may generate the information related to the head movement speed and the eye movement speed within a preset time based on a time when the head movement becomes greater than or equal to a preset threshold value.

The balance function status information generator may calculate a gain using a time value at which the head movement speed is maximum and a time value at which the eye movement speed is maximum.

When n is greater than or equal to 2, the head movement generator may further generate reference head movement information by calculating statistical values of the information related to the head coordinates according to the m-th video, and the eye movement generator may further generate reference eye movement information by calculating statistical values of the information related to the coordinates of the pupil center and the eye phase changes according to the m-th video.

The at least one processor may include: a head movement generator that generates the information related to head movement in the m-th (natural number from 1 to n) video using the information related to the head coordinates acquired from at least one artificial neural network model; an eye movement generator that generates the eye movement information in the m-th video using the information related to the coordinates of a pupil center or the eye phase changes acquired from the at least one artificial neural network model; a target output generator that outputs a virtual target to a display device; a head direction provider that provides direction information on the head movement to the subject; and a feedback provider that provides feedback according to the head movement and the eye movement of the subject.

A method of generating information on a balance function status according to another exemplary embodiment of the present specification in a balance function management system including at least one processor and a memory that stores instructions executable by the processor and stores at least one artificial neural network model executed on a computing device may include: acquiring, by the at least one processor, information related to head coordinates, coordinates of a pupil center, and eye phase changes of the subject according to an order of frame images of an m-th (natural number from 1 to n) video by allowing the at least one processor to input frame images of n videos of a subject captured by n (natural number) cameras to at least one artificial neural network model; and generating, by the at least one processor, head movement information and eye movement information using the information acquired from the at least one artificial neural network model, calculating a head movement speed and an eye movement speed, and generating information related to a balance function status using information related to the head movement speed and the eye movement speed.

A balance function rehabilitation method according to still another exemplary embodiment of the present specification in a balance function management system including at least one processor and a memory that stores instructions executable by the processor and stores at least one artificial neural network model executed on a computing device may include: acquiring, by the at least one processor, information related to head coordinates, coordinates of a pupil center, and eye phase changes of the subject according to an order of frames of an m-th (natural number from 1 to n) video by allowing the at least one processor to input frame images of n videos of a subject captured by n (natural number) cameras to at least one artificial neural network model; and generating, by the at least one processor, head movement information and eye movement information using the information acquired from the at least one artificial neural network model and performing a balance function rehabilitation program by tracking head and eye movements of a subject.

The method of generating information on a balance function status according to the present specification may be implemented in the form of a computer program written to perform the method on a computer and recorded on a computer-readable recording medium.

The balance function rehabilitation method according to the present specification may be implemented in the form of a computer program written to perform the method on a computer and recorded on a computer-readable recording medium.

Other detailed contents of the present disclosure are described in a detailed description and are illustrated in the drawings.

### Effect of the Invention

According to one aspect of the present specification, the balance function management system can input the video in which the subject performing the balance function status check is captured using the smart phone, the webcam, etc., to the artificial neural network model to calculate the eye movement and head movement without separate medical equipment and generate the information on the balance function status.

According to another aspect of the present specification, the balance function management system can be utilized as the clinical decision support system for the dizzy patient that generates the information on the balance function status.

According to another aspect of the present specification, the balance function management system can provide the important information for quickly distinguishing the peripheral and central dizziness with only the single smart phone without using the separate medical device in the emergency room environment or without the patient wearing the separate device.

According to another aspect of the present specification, the balance function management system can provide the remote balance function rehabilitation program by outputting the movement information on patient's head position change and eye movement to the display device to enable more accurate balance rehabilitation.

Effects of the present disclosure are not limited to the effects described above, and other effects that are not mentioned may be obviously understood by those skilled in the art from the following description.

The effects of the present disclosure are not limited to the aforementioned effects, and other effects, which are not mentioned above, will be apparently understood to a person having ordinary skill in the art from the following description.

The objects to be achieved by the present disclosure, the means for achieving the objects, and the effects of the present disclosure described above do not specify essential features of the claims, and, thus, the scope of the claims is not limited to the disclosure of the present disclosure.

### BRIEF DESCRIPTION OF DRAWINGS

The above and other aspects, features and other advantages of the present disclosure will be more clearly understood from the following detailed description taken in conjunction with the accompanying drawings, in which:

FIG. 1 is a reference diagram illustrating a scene of capturing a subject with at least one camera;

FIG. 2 is a reference diagram illustrating a scene of a subject captured by a camera included in a terminal;

FIG. 3 is a block diagram of a balance function management system according to a first exemplary embodiment of the present specification;

FIG. 4 is an example of a scene of capturing a subject that performs a balance function status check and/or a balance function rehabilitation program according to an exemplary embodiment of the present specification;

FIG. 5 is a diagram illustrating an example of a scene of capturing a subject that performs a balance function status check and/or a balance function rehabilitation program according to another exemplary embodiment of the present specification;

FIG. 6 is a diagram illustrating an example of preprocessing an eye area image according to an exemplary embodiment of the present specification;

FIG. 7 is a diagram illustrating an example of generating training data of a second artificial neural network model according to another exemplary embodiment of the present specification;

FIG. 8 is a block diagram of a balance function management system for generating information on a balance function status according to an embodiment exemplary of the present specification;

FIG. 9 is a diagram illustrating an example of outputting information on a balance function status to a display according to an exemplary embodiment of the present specification;

FIG. 10 is a block diagram of a balance function management system for performing a balance function rehabilitation program according to embodiment an exemplary of the present specification;

FIG. 11 is a diagram illustrating an example of a scene performing the balance function rehabilitation program according to an exemplary embodiment of the present specification;

FIG. 12 is a block diagram of a balance function management system for generating information on a balance function status and performing a balance function rehabilitation program according to an exemplary embodiment of the present specification;

FIG. 13 is a block diagram of a balance function management system according to a second exemplary embodiment of the present specification;

FIG. 14 is a reference diagram illustrating an example in which a head coordinate learning unit concatenates frame images according to an exemplary embodiment of the present specification;

FIG. 15 is a diagram illustrating an example of a multi-frame image of a scene where a subject performing a balance function status check and/or a balance function rehabilitation program according to an exemplary embodiment of the present specification is captured;

FIG. 16 is a diagram illustrating an example of the multi-frame image of the scene where the subject performing the balance function status check and/or the balance function rehabilitation program according to an exemplary embodiment of the present specification is captured;

FIG. 17 is a diagram illustrating an example of preprocessing an eye area image according to an exemplary embodiment of the present specification;

FIG. 18 is a block diagram of a balance function management system for generating information on a balance function status according to an exemplary embodiment of the present specification;

FIG. 19 is a block diagram of a balance function management system for performing a balance function rehabilitation program according to an exemplary embodiment of the present specification;

FIG. 20 is a block diagram of a balance function management system for generating information on a balance function status and performing a balance function rehabilitation program according to an exemplary embodiment of the present specification;

FIG. 21 is a flowchart of a method of generating information on a balance function status according to an exemplary embodiment of the present specification; and

FIG. 22 is a flowchart of a balance function rehabilitation method according to an exemplary embodiment of the present specification.

### DETAILED DESCRIPTION OF THE EMBODIMENT

Hereinafter, the exemplary embodiment of the present disclosure will be described with reference to the accompanying drawings and exemplary embodiments as follows. Scales of components illustrated accompanying drawings are different from the real scales for the purpose of description, so that the scales are not limited to those illustrated in the drawings.

Various advantages and features of the present disclosure disclosed in the present specification and methods accomplishing them will become apparent from the following description of exemplary embodiments with reference to the accompanying drawings. However, the present specification is not limited to exemplary embodiments to be described below, but may be implemented in various different forms, these exemplary embodiments will be provided only in order to make the disclosure of the present specification complete and allow those skilled in the art (hereinafter referred to as ‘those skilled in the art’) to which the present specification pertains to completely recognize the scope of the present specification, and the scope of rights in the present specification is only defined by the scope of the claims.

Terms used in the present specification are for describing exemplary embodiments rather than limiting the scope of rights of the present specification. Unless explicitly described to the contrary, a singular form includes a plural form in the present specification. Terms “comprise/include” and/or “comprising/including” used in the present specification do not exclude the existence or addition of one or more other components other than the mentioned components.

Throughout the present specification, the same components will be denoted by the same reference numerals, and a term “and/or” includes each and all combinations of one or more of the mentioned components. Terms “first”, “second” and the like are used to describe various components, but these components are not limited by these terms. These terms are used only in order to distinguish one component from other components. Accordingly, a first component mentioned below may be a second component within the technical spirit of the present disclosure.

Unless defined otherwise, all terms (including technical and scientific terms) used in the present specification have the same meanings commonly understood by those skilled in the art to which the present specification pertains. In addition, terms defined in generally used dictionaries are not ideally or excessively interpreted unless they are specifically defined clearly.

An artificial neural network (ANN) implements artificial intelligence by connecting artificial neurons that are mathematically modeled with neurons that make up a human brain.

An “artificial neural network model” in the present specification generally be composed of a set of interconnected computational units, which may be referred to as nodes. These “nodes” may also be referred to as “neurons.” The neural network is configured to include at least one node. The nodes (or neurons) that constitute a neural network may be interconnected by one or more links.

Within the neural network, one or more nodes connected through the link may form a relative relationship between the input node and the output node. The concepts of the input node and the output node are relative, and any node in the relationship of the output node with respect to one node may be in the input node relationship in the relationship with another node, and vice versa. As described above, the relationship between the input node and the output node may be generated around the link. One or more output nodes may be connected to one input node through the link, and vice versa.

First input nodes may refer to one or more nodes, to which data is directly input without going through links in relationships with other nodes, among the nodes in the neural network. Alternatively, the first input nodes may refer to nodes that do not have other input nodes connected by the link in the relationship between the nodes based on the link within the neural network. Similarly, the final output nodes may refer to one or more nodes that do not have the output node in the relationship with other nodes among the nodes in the neural network. In addition, hidden nodes may refer to nodes that constitute the neural network rather than the first input node and the last output node.

In the present specification, “inputting” data into an artificial neural network model refers to that any value is input to the first input node. In the present specification, “acquiring a value,” “outputting data,” “acquiring information,” etc., from the artificial neural network refer to that any data is output from the last output node.

A deep neural network (DNN) may refer to a neural network including a plurality of hidden layers in addition to an input layer and an output layer. The deep neural network may include a convolutional neural network (CNN), a recurrent neural network (RNN), an auto encoder, generative adversarial networks (GAN), a restricted Boltzmann machine (RBM), a deep belief network (DBN), a Q network, a U network, a Siamese network, a generative adversarial network (GAN), and the like. The description of the deep neural network described above is only an example, and the present disclosure is not limited thereto.

The neural network may be trained in at least one of supervised learning, unsupervised learning, semisupervised learning, or reinforcement learning. The training of the neural network may be a process of applying knowledge for the neural network to perform a specific operation to the neural network.

The neural network may be trained in a direction that minimizes an output error. In the training of the neural network, a process of repeatedly inputting training data to the neural network, calculating an output of the neural network for the training data and target errors, and updating weights of each node of the neural network by backpropagating errors of the neural network from an output layer of the neural network to an input layer in order to reduce the errors may be performed. In the case of the supervised learning, training data with a correct answer labeled for each training data is used (i.e., labeled training data), and in the case of the unsupervised learning, a correct answer may not be labeled for each training data. That is, for example, in the case of the supervised learning for data classification, the training data may be data in which each category is labeled for training data. The labeled training data is input to the neural network, and an error may be calculated by comparing the output (category) of the neural network with the label of the training data. As another example, in the case of the unsupervised learning for the data classification, the error may be calculated by comparing the input training data with the output of the neural network. The calculated error is backpropagated in a backward direction (i.e., from an output layer to an input layer) in the neural network, and connection weights of each node in each layer of the neural network may be updated according to the backpropagation. The amount of change in the connection weights of each node to be updated may be determined according to a learning rate. The calculation of the neural network for the input data and the backpropagation of the error may constitute a learning cycle (epoch). The learning rate may be applied differently depending on the number of times of repetitions of the learning cycle of the neural network. For example, in the early stage of the training of the neural network, a high learning rate may be used to allow the neural network to quickly acquire a certain level of performance, thereby increasing efficiency, and in the later stage of the training, a low learning rate may be used to increase accuracy.

In the present specification, the “training” of the artificial neural network model refers to that the neural network updates the connection weights of each node so that the output error is minimized, and the “training” according to the present specification is not limited to a specific training method.

In the present specification, the “processor” may be composed of one or more cores, and may include a processor for data analysis and deep learning, such as a central processing unit (CPU), a general purpose graphics processing unit (GPGPU), a tensor processing unit (TPU), etc., of a computing device. The processor may read a computer program stored in a memory and perform data processing for machine learning according to an exemplary embodiment of the present specification. According to an exemplary embodiment of the present specification, the processor may perform calculations for training the neural network. The processor may perform calculations for training a neural network, such as processing input data for training in deep learning (DL), extracting features from the input data, calculating errors, and updating weights of the neural network using backpropagation. The processor may allow at least one of CPU, GPGPU, and TPU to process the training of the network function. For example, both the CPU and GPGPU may process the training of the network function and the data classification using the network function. In addition, in an exemplary embodiment of the present specification, processors of a plurality of computing devices may be used together to process training of a network function and data classification using the network function. In addition, a computer program executed in a computing device according to an exemplary embodiment of the present specification may be a CPU, GPGPU, or TPU executable program.

Hereinafter, exemplary embodiments of the present disclosure will be described in detail with reference to the accompanying drawings.

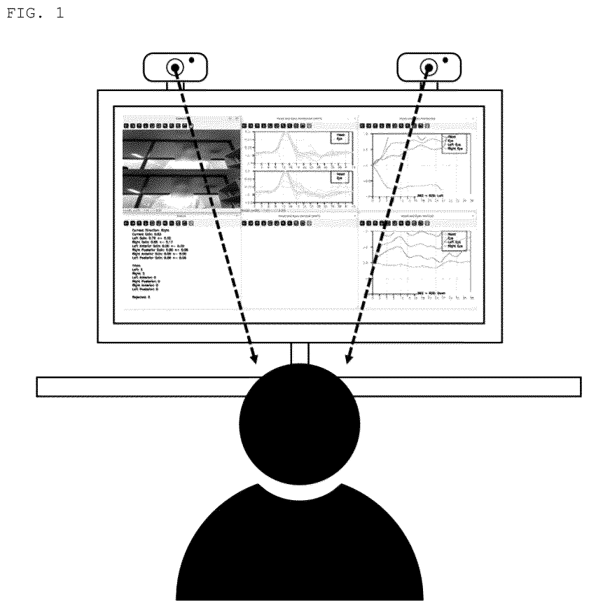

FIG. 1 is a reference diagram illustrating a scene of capturing a subject with at least one camera.

Referring to FIG. 1, a balance function management system according to exemplary an embodiment of the present specification may generate head movement and eye movement information of a subject using n videos of the subject captured by n (a natural number) cameras.

The n cameras may be installed at preset positions to capture a face of a subject. The n cameras may capture a subject at different angles.

For example, one camera may be installed at the front of the subject to capture the face of the subject. The front of the subject may refer to a direction in which a front of a body faces.

As another example, two cameras may be installed at preset positions to capture the face of the subject. In this case, one of the two cameras may capture the face of the subject from the left of the front of the subject, and the other camera may capture the face of the subject from the right of the front of the subject.

As another example, three cameras may be installed at preset positions to capture the face of the subject. In this case, one of the three cameras may be installed in the front of the subject, the other may be installed on the left of the front of the subject, and another may be installed on the right of the front of the subject to capture the face of the subject.

The number of cameras and the positions of the cameras correspond to an example, and are not limited thereto. Depending on the number of cameras, the positions of the cameras, the distance of the cameras from the subject, etc., n cameras may capture the subject from various directions and distances.

The balance function management system may receive video data of the subject captured by the n cameras. The balance function management system may display the received video data on a display device. The balance function management system may display m-th videos of a subject captured by an m-th (natural number from 1 to n) camera on the display device, respectively. Hereinafter, the m-th video may refer to all videos from a first video to an n-th video.

The balance function management system may generate information related to head and eye movements of the subject using the received n input videos. The balance function management system may display the information related to the head and eye movements on the display device. In addition, the balance function management system may generate information related to head and eye movement speeds using the information related to the head and eye movements, and display the generated information on the display device.

The screen displayed on the display device of FIG. 1 corresponds to an example and the present disclosure is not limited to the screen.

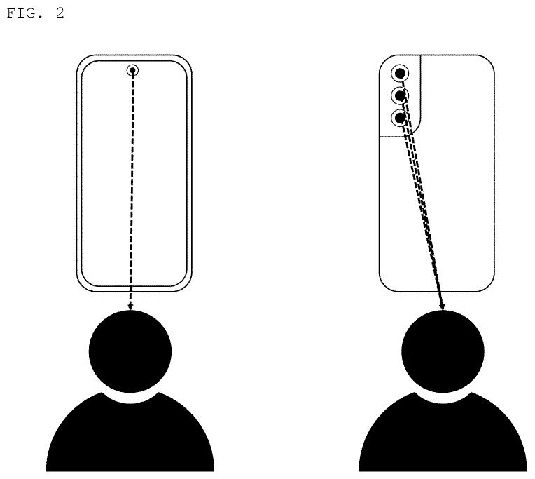

FIG. 2 is a reference diagram illustrating a scene of a subject captured by a camera included in a terminal.

Referring to FIG. 2, according to another exemplary embodiment of the present specification, the balance function management system may generate information related to head and eye movements using a video captured by a front camera (left of FIG. 2) and/or a rear camera (right of FIG. 2) of a terminal such as a smart phone or a tablet computer. When capturing the subject using the rear camera of the terminal, the balance function management system may display each video captured by at least one camera included in the rear of the terminal on the display device. The balance function management system may generate information related to the head and eye movements of the subject from each video captured by at least one camera included in the rear of the terminal.

The video captured by the front camera and/or the rear camera of the terminal may be displayed on the display of the terminal and/or the display device connected to the terminal.

The number, positions, and directions of front cameras and/or rear cameras of the terminal illustrated in FIG. 2 correspond to an example, and the present disclosure is not limited thereto. In addition, at least one camera for capturing the subject may be installed.

The balance function management system according to the present specification may be implemented in the form of a computing device such as a computer, a laptop, a smart phone, and/or a tablet computer, which corresponds to an example, and the present disclosure is not limited to the device.

The subject may be captured by the camera included in the computing device and/or the camera connected to the computing device in a wired and/or wireless manner. Various types of cameras such as a webcam and an action camera may be used as the camera, which corresponds to an example, and the present disclosure is not limited to the camera.

The balance function management system may generate information related to the balance function status of the subject by using n videos of the subject performing a video head impulse test, a spontaneous nystagmus test, a saccade test, etc., which corresponds to an example, and the present disclosure is not limited to the tests.

Hereinafter, a balance function management system according to a first exemplary embodiment of the present specification will be described. According to the first exemplary embodiment of the present specification, the balance function management system may generate information on head and eye movements of a subject using at least one video of the subject captured by at least one camera to generate information on a balance function status and/or perform a balance function rehabilitation program.

The balance function management system according to the first exemplary embodiment of the present specification may include at least one processor and a memory that stores instructions executable by the processor and stores at least one artificial neural network model executed on a computing device.

The balance function management system may allow the at least one processor to input frame images of n videos of the subject captured by n cameras to at least one artificial neural network model, and acquire at least one of information related to head coordinates, coordinates of pupil center, and eye phase changes of the subject according to the order of the frame images. The at least one processor may use the information to generate head movement information and eye movement information.

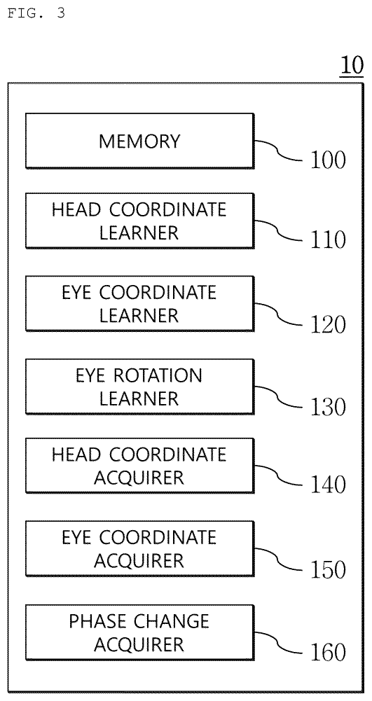

FIG. 3 is a block diagram of the balance function management system according to the first exemplary embodiment of the present specification.

Referring to FIG. 3, a balance function management system 10 according to the first exemplary embodiment of the present specification may include a memory 100, a head coordinate learner 110, an eye coordinate learner 120, an eye rotation learner 130, a head coordinate acquirer 140, an eye coordinate acquirer 150, and a phase change acquirer 160.

The memory 100 may store at least one of a first artificial neural network model that generates information related to head coordinates, a second artificial neural network model that acquires information related to coordinates of a pupil center, and a third artificial neural network model that acquires information related to eye phase changes.

According to an exemplary embodiment of the present specification, the first artificial neural network model may be trained by allowing the head coordinate learner 110 to use facial feature points and coordinates of the feature points extracted from at least one frame image of a video in which a human face is captured as training data.

The head coordinate learner 110 may train the first artificial neural network model using at least one video in which a human face is captured. At least one video in which the human face is captured may be a video in which an appearance in which the captured human head rotates is captured.

For example, the video in which the human face is captured may be captured by a single camera. The video in which the human face is captured may include an image of the entire human face. The video in which the human face is captured may be a video in which a scene where a head moves left and right is captured while the captured human face faces forward. In addition, the video in which the human face is captured may be a video in which a scene where a head moves up and down is captured while the captured human face faces forward. The state in which the human face faces forward may be a state in which a gaze direction of a person and a front direction of a body are consistent.

In addition, the video in which the human face is captured may be a video in which the scene where the head moves up and down is captured after the captured human head is turned to the right. In addition, the video in which the human face is captured may be a video in which the scene where the head moves up and down is captured after the captured human head is turned to the left.

In addition, the video in which the human face is captured may be a video in which the scene where the head rotates is captured while the captured human gazes at a specific gaze point.

The video in which the human face is captured may be captured by a camera installed in the front of the person. Alternatively, the video in which the human face is captured may be captured by cameras installed at various angles at various positions such as the left, right, upper left, and upper right of the front of the person.

In addition, the video in which the human face is captured may be captured by a plurality of cameras. The plurality of cameras may be installed at various angles at various positions to capture a person. The video captured by the plurality of cameras may refer to a video in which the plurality of cameras captures the same situation.

The video in which the human face is captured may be captured by cameras in which all the specifications of the cameras are the same. In addition, the video in which the human face is captured may be a video that is captured by cameras having different specifications. In addition, the video in which the human face is captured may be a video that is captured by cameras having different setting values.

The above-described video in which the person is captured corresponds to an example, and the video in which the human head and eye are captured or all the videos in which the human head is captured may be used. In addition, the present disclosure is not limited to specific camera specifications, settings, etc.

Preferably, the video in which the person is captured may be a video captured by a camera having the same setting value.

The head coordinate learner 110 may extract feature points for a human face from each frame image of the video in which the human face is captured. For example, the head coordinate learner 110 may extract feature points positioned on a human face from each frame image. For example, the feature points may include feature points for a tip of a nose, a left outer canthus (point where an outermost eyelid of a left eye meets), a right outer canthus (point where an outermost eyelid of a right eye meets), and a forehead of a person. The positions of the feature points may not change even if a person blinks. The feature points correspond to an example, and the present disclosure is not limited to the feature points, and feature points used for conventional face recognition may be further included. Since the technology for generating feature points and feature point coordinates from a human head is a technology widely known to those skilled in the art, a detailed description thereof will be omitted.

The head coordinate learner 110 may generate coordinate information of feature points extracted from each frame image. The head coordinate learner 110 may train a first artificial neural network model to generate three-dimensional head coordinates for each frame image using the feature points and coordinates of the feature points extracted from each frame image as training data. The 3D head coordinates may refer to 3D coordinates of a head according to a 3D standard head model. The 3D coordinates of the head may include coordinates for all feature points of the head. The 3D coordinates of the head may include contents related to an index number for each feature point. In this case, the head coordinate learner 110 may train the first artificial neural network model to generate the index number based on the feature points of the tip of the nose. In addition, the head coordinate learner 110 may also be trained to further generate head movement information using 3D coordinates extracted from each frame image.

In addition, the head coordinate learner 110 may train the first artificial neural network model further using, as training data, data on a 3D standard head model, such as the frame image from which the feature points are extracted, a 3D morphable model (3DMM), a faces learned with an articulated model and expressions (FLAME) model, which corresponds to an example, and the present disclosure is not limited to the training data, and various training data may be additionally used.

According to an exemplary embodiment of the present specification, the head coordinate learner 110 may extract feature points after adjusting the sync of the multiple videos when extracting the feature points from the multiple videos in which a person is captured using a plurality of cameras. The head coordinate learner 110 may adjust the sync of the multiple videos using an algorithm such as a specific audio signal of the multiple videos and feature point matching-based synchronization, which corresponds to an example, and the present disclosure is not limited thereto, and various technologies widely known to those skilled in the art may be used.

According to an exemplary embodiment of the present specification, the head coordinate learner 110 may use feature points having coordinate values according to preset criteria as training data. The head coordinate learner 110 may select the feature points having the coordinate values according to the preset criteria as training data.

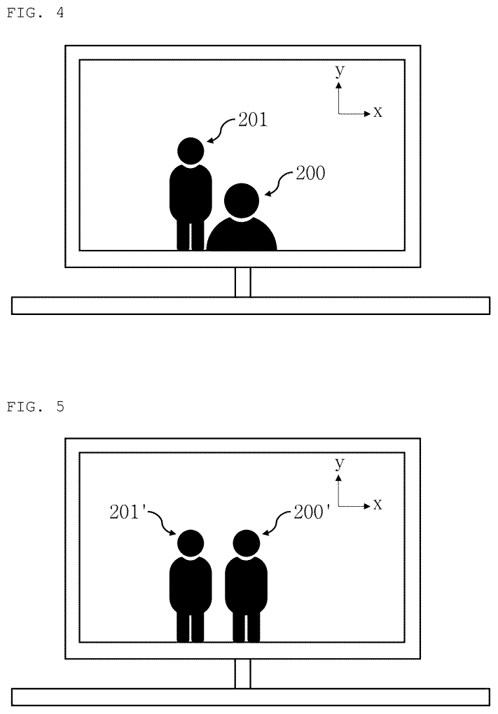

FIG. 4 is an example of a scene of capturing a subject that performs a balance function status check and/or a balance function rehabilitation program according to an exemplary embodiment of the present specification.

Referring to FIG. 4, the video in which the person is captured may be a video of performing the balance function status check and/or the balance function rehabilitation program using the balance function management system 10. The video may be a video in which only a subject 200 is captured. In addition, the video may be a video in which the subject 200 and an examiner 201 are captured. Hereinafter, the video in which the person is captured will be described as meaning the video of performing the balance function status check and/or the balance function rehabilitation program. However, this corresponds to an example and the present disclosure is not limited to the video.

When performing the balance function status check, the subject 200 may be sitting on a chair and the examiner 201 may be standing behind the subject. The examiner 201 may refer to a medical professional. The balance function management system 10 may analyze only the head movement and the eye movement of the subject 200 to determine whether the balance function of the subject is abnormal by analyzing the eye movement according to the head movement of the subject 200 and to proceed with the rehabilitation program.

The head coordinate learner 110 may extract the feature points and the coordinates of the feature points for the face of the subject 200 from the video to train the first artificial neural network model.

As described above, since the subject 200 is sitting on a chair and the examiner 201 is standing, the feature points extracted from the face of the subject 200 may be positioned relatively lower than the feature points extracted from the face of the examiner 201. This may refer to that a y-coordinate value of the feature points extracted from the face of the subject 200 is relatively smaller than a y-coordinate value of the feature points extracted from the face of the examiner 201.

The head coordinate learner 110 may extract, as training data, feature points with relatively smaller y-coordinate values among feature points of the same portion extracted from the faces of the subject 200 and the examiner 201. The head coordinate learner 110 may extract feature points positioned in an area below the preset y-coordinate value as training data.

In addition, the head coordinate learner 110 may cluster feature points extracted from the faces of the subject 200 and the examiner 201. Thereafter, the head coordinate learner 110 may extract a set of feature points positioned at a relatively lower side as training data.

FIG. 5 is a diagram illustrating an example of a scene of capturing a subject that performs a balance function status check and/or a balance function rehabilitation program according to another exemplary embodiment of the present specification.

Referring to FIG. 5, in the video, the balance function status check and/or the balance function rehabilitation program may be performed while both the subject 200′ and examiner 201′ are standing. In this case, the feature points extracted from the face of the subject 200′ may be relatively closer to the center of the display screen than the feature points extracted from the face of the examiner 201′. The head coordinate learner 110 may extract the feature points having the coordinate values that are relatively closer to the center of the display screen as training data.

In another example, the subject may be relatively closer to the camera than the examiner. As a result, the face of the subject may occupy a relatively wider area than the face of the examiner in the video. The head coordinate learner 110 may extract, as training data, a set of feature points which are distributed relatively over a wider area, among a set of feature points extracted from the face of the subject and a set of feature points extracted from the face of the examiner.

The process in which the head coordinate learner 110 described above extracts only the feature points extracted from the face of the subject as training data is an example, and the present disclosure is not limited thereto, and various criteria may be set according to the position of the subject, the position of the examiner, the position of the camera, the angle, etc. In addition, only the feature points extracted from the face of the subject may be extracted as training data through markers, etc., for distinguishing the subject and the examiner as well as the positions of the faces of the subject and the examiner. Therefore, various exemplary embodiments may arise depending on various situations in which the balance function status check and/or the rehabilitation examination program are performed.

In the video, when the head of the subject quickly rotates, the face of the subject may not be clearly captured in the frame image. In this case, the head coordinate learner 110 may not extract the feature points from the face of the subject. The head coordinate learner 110 may train the first artificial neural network model using the feature points extracted from the face of the examiner, so the first artificial neural network model may produce inaccurate results.

According to an exemplary embodiment of the present specification, the head coordinate learner 110 may track the coordinates of the feature points extracted from the frame image preceding an arbitrary frame image. As described above, when the feature points are not extracted from the face of the subject in the arbitrary frame image, the head coordinate learner 110 may extract the training data using the image of the subject in the preceding frame image. The preceding frame image may refer to the closest frame image from which feature points may be extracted from the face of the subject among the frame images preceding the arbitrary frame image.

According to an exemplary embodiment of the present specification, the second artificial neural network model may be trained to generate information related to coordinates of a pupil center by allowing the eye coordinate learner 120 to use, as training data, an eye area image, which is an image of an area including an eye in a video frame image in which a human face is captured.

The eye coordinate learner 120 may extract an eye area image of a subject, which is an image of an area including an eye of the subject, from the video. The eye coordinate learner 120 may train the second artificial neural network model to generate the information related to the coordinates of the pupil center by using the eye area image of the subject extracted from each frame image as training data.

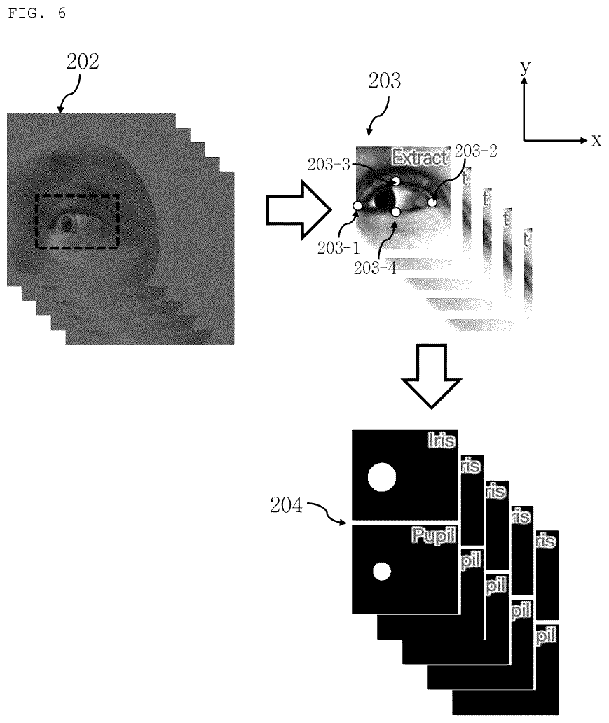

FIG. 6 is a diagram illustrating an example of preprocessing an eye area image according to an exemplary embodiment of the present specification.

Referring to FIG. 6, the eye coordinate learner 120 may extract an eye area image 203 according to each frame image 202. The eye coordinate learner 120 may extract an image inside a bounding box of an eye area from each frame image 202 as the eye area image 203. The eye coordinate learner 120 may segment the iris and pupil areas from the eye area image 203. Since the technology of extracting the eye area from the human face and segmenting the iris and pupil areas is a technology widely known to those skilled in the art, a detailed description thereof will be omitted.

The eye coordinate learner 120 may estimate an area for a part where the iris and/or pupil are covered by an eyelid. As illustrated in FIG. 6, a part of the iris may be covered by an upper eyelid and a lower eyelid. The eye coordinate learner 120 may estimate the covered part using an ellipse fitting algorithm, a circle Hough transform algorithm, or the like. Alternatively, the eye coordinate learner 120 may segment the iris and pupil areas using the artificial neural network model that has been previously trained to segment the iris and pupil areas. This corresponds to an example and the present disclosure is not limited to the method.

The eye coordinate learner 120 may train the second artificial neural network model using data in which the iris and/or pupil areas are segmented in the eye area image 203. For example, the eye coordinate learner 120 may segment the iris and/or pupil areas in the eye area image 203 to generate a mask image 204. The eye coordinate learner 120 may generate a mask image in which the area occupied by the iris and/or pupil and the remaining area have different pixel values in the eye area image 203. The mask image may be displayed in white or black for the area occupied by the iris and/or pupil, and displayed in black or white for the remaining area, which is an example, and the present disclosure is not limited thereto.

The eye coordinate learner 120 may train the second artificial neural network model to generate the information related to the coordinates of the pupil center using the mask image 204 as training data. Alternatively, the eye coordinate learner 120 may train the second artificial neural network model using two-dimensional pixel values of the mask image 204 as training data. Alternatively, the eye coordinate learner 120 may train the second artificial neural network model using the mask image 204 and the two-dimensional pixel values as training data.

As another example, the eye coordinate learner 120 may train the second artificial neural network model using a heatmap model that segments and displays the iris and/or pupil area in the eye area image 203.

The eye coordinate learner 120 may train the second artificial neural network model using at least one of the mask image and the heatmap model.

According to an exemplary embodiment of the present specification, the eye coordinate learner 120 may train the second artificial neural network model to generate the eye feature points and coordinate information of the feature points from the eye area image 203, and to generate the horizontal coordinate values and vertical coordinate values of the pupil center using the coordinates of the plurality of preset feature points. The eye coordinate learner 120 may extract normalized coordinates of a pupil center using the coordinates of the plurality of feature points extracted from the eye area image 203.

The coordinates of the plurality of feature points may include a feature point having a relatively smallest x-axis coordinate value and a feature point having a relatively largest x-axis coordinate value among feature points whose y-axis coordinates are within a preset range in the eye area image 203.

The coordinates of the plurality of feature points may include a feature point having a relatively smallest y-axis coordinate value and a feature point having a relatively largest y-axis coordinate value among feature points whose x-axis coordinates are within a preset range in the eye area image 203 captured from the front of the person.

For example, the eye coordinate learner 120 may extract horizontal coordinates of a normalized pupil center using a feature point 203-1 for a medial canthus and a feature point 203-2 for an outer canthus among the feature points extracted from the eye area image 203. The eye coordinate learner 120 may use a line segment connecting the feature point 203-1 for the medial canthus and the feature point 203-2 for the outer canthus as a horizontal axis for the coordinates of the pupil center. The x-coordinate of the feature point 203-1 for the medial canthus and the x-coordinate of the feature point 203-2 for the outer canthus may correspond to both extreme values of the horizontal axis. The difference between the x-coordinate of the feature point 203-1 for the medial canthus and the x-coordinate of the feature point 203-2 for the outer canthus may refer to the entire length of the horizontal axis. The eye coordinate learner 120 may calculate the horizontal coordinates of the normalized pupil center using the horizontal coordinate values of the pupil center compared to the length of the entire horizontal axis.

In addition, the eye coordinate learner 120 may extract vertical coordinates of the normalized pupil center using feature points related to upper and lower eyelids among the feature points extracted from the eye area image 203. In this case, as the feature point related to the upper eyelid, a feature point 203-3 with the largest y-axis coordinate among the feature points extracted from the eye may be used. Hereinafter, the feature point 203-3 will be referred to as an upper eyelid feature point.

As the feature point related to the lower eyelid, a feature point 203-4 with the smallest y-axis coordinate among the feature points extracted from the eye may be used. Hereinafter, the feature point 203-4 will be referred to as a lower eyelid feature point.

The eye coordinate learner 120 may use a line segment connecting the upper eyelid feature point 203-3 and the lower eyelid feature point 203-4 as a vertical axis for the coordinates of the pupil center. The y-coordinate of the upper eyelid feature point 203-3 and the y-coordinate of the lower eyelid feature point 203-4 may correspond to the two extreme values of the vertical axis. The difference between the y-coordinate of the upper eyelid feature point 203-3 and the y-coordinate of the lower eyelid feature point 203-4 may refer to the length of the entire vertical axis. The eye coordinate learner 120 may calculate the horizontal coordinates of the normalized pupil center using the vertical coordinate values of the pupil center compared to the length of the entire vertical axis. This corresponds to an example and the present disclosure is not limited to the feature point.

In FIG. 6, the feature point 203-1 for the medial canthus and the feature point 203-2 for the outer canthus are illustrated as being positioned on the same horizontal line, and the upper eyelid feature point 203-3 and the lower eyelid feature point 203-4 are illustrated as being positioned on the same vertical line. However, this may vary depending on a capturing angle of a camera, a head angle of a persona, etc.

For example, in the case of the eye area image rotating 30° clockwise based on the eye area image 203, the feature point 203-1 for the medial canthus and the feature point 203-2 for the outer canthus may not be positioned on the same horizontal line, and the upper eyelid feature point 203-3 and the lower eyelid feature point 203-4 may not be positioned on the same vertical line. In this case, the eye coordinate learner 120 may transform the image so that in the rotating eye area image, the feature point 203-1 for the medial canthus and the feature point 203-2 for the outer canthus are positioned on the same horizontal line, and the upper eyelid feature point 203-3 and the lower eyelid feature point 203-4 are positioned on the same vertical line. In this case, the eye coordinate learner 120 may transform the rotating eye area image using an affine transform, etc., and this is an example, and the present disclosure is not limited thereto.

The eye coordinate learner 120 may train the second artificial neural network model further using the horizontal coordinate values and the vertical coordinate values of the generated normalized pupil center as training data.

In addition, the eye coordinate learner 120 may generate the horizontal coordinate values and the vertical coordinate values of the pupil center using frame images of multiple videos captured by a plurality of cameras. The multiple videos may be synchronized videos. The eye coordinate learner 120 may calculate the average value of the horizontal coordinate values and the vertical coordinate values of the pupil center calculated from the multiple frame images whose synchronization matches each other in the multiple videos. The eye coordinate learner 120 may train the second artificial neural network model further using the average value of the horizontal coordinate values and the vertical coordinate values of the pupil center as training data. Accordingly, the second artificial neural network model may generate information related to more accurate coordinates of a pupil center. The information related to the coordinates of the pupil center may include the vertical coordinate values and the horizontal coordinate values of the pupil center, the movement information of the pupil center in the vertical direction and the movement information of the pupil center in the horizontal direction, etc., according to each frame image. The information related to the coordinates of the pupil center may include contents about the two-dimensional coordinates.

The eye coordinate learner 120 may extract the eye area images for the left and right eyes from the frame images, and train the second artificial neural network model to generate the information related to the coordinates of the pupil center of the left eye and the information related to the coordinates of the pupil center of the right eye.

According to another exemplary embodiment of the present specification, the memory 100 may store data of at least one virtual object. The second artificial neural network model may be trained to generate information related to the coordinates of the pupil center by allowing the eye coordinate learner 120 to use training data that includes a parameter value obtained by changing at least one of parameters related to head rotation, eye rotation, and camera settings of a parameter of a virtual object and an image of the virtual object acquired according to the parameter value.

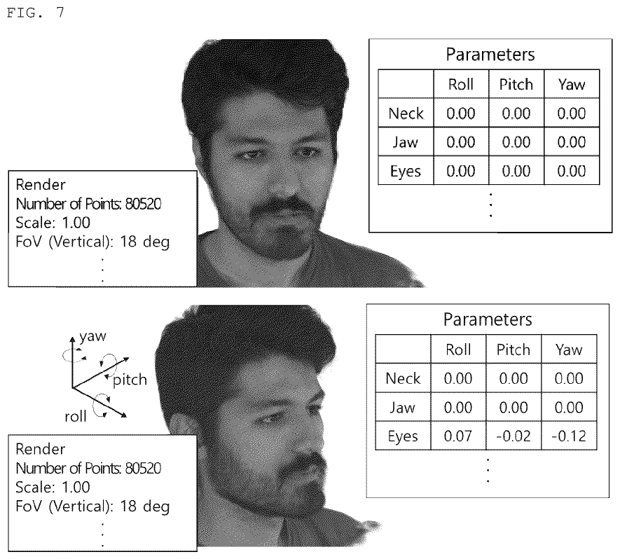

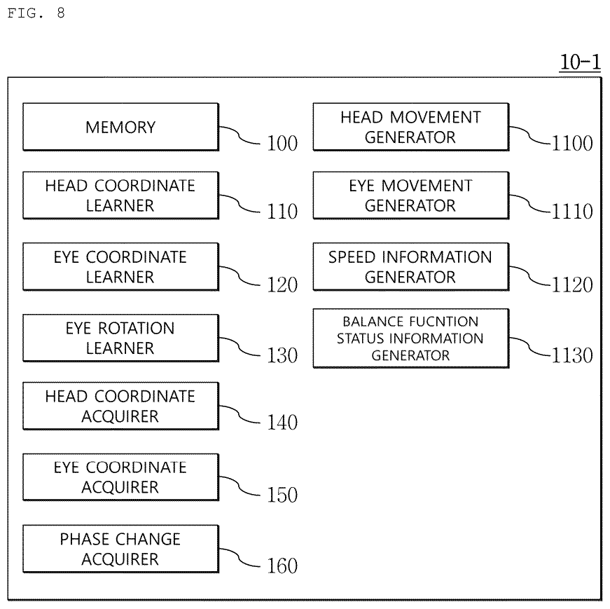

FIG. 7 is a diagram illustrating an example of generating training data of a second artificial neural network model according to another exemplary embodiment of the present specification.

Referring to FIG. 7, the eye coordinate learner 120 may change at least one of the parameters related to the head rotation, the eye rotation, and the camera settings of the virtual object. As an example, the eye coordinate learner 120 may set parameter values so that the virtual camera captures the virtual object from the front (upper drawing of FIG. 7). The eye coordinate learner 120 may change parameter values so that the virtual camera captures the virtual object from the right (lower drawing of FIG. 7). In addition, the eye coordinate learner 120 may change parameter values for a distance between the virtual camera and the virtual object.

The eye coordinate learner 120 may set parameter values so that the head of the virtual object rotates in at least one direction of a roll, a pitch, and a yaw.

The eye coordinate learner 120 may set parameter values so that the eye of the virtual object rotates in at least one direction of the roll, the pitch, and the yaw.

The eye coordinate learner 120 may change at least one of the parameters and acquire the image of the virtual object. The eye coordinate learner 120 may train the second artificial neural network model using the parameter values and the virtual object according to the parameter values. In this case, the second artificial neural network model may be trained to generate the information on the two-dimensional coordinates and/or three-dimensional coordinates of the pupil center.

The virtual object may mean a Gaussian avatar generated using a 3D Gaussian splatter. The eye coordinate learner 120 may control a latent vector of the virtual object to change Euler coordinates of the head and pupil of the virtual object. The Gaussian avatar corresponds to an example, and the present disclosure is not limited thereto, and a virtual object generated using a technique widely known among those skilled in the art may be used.

In addition, the memory 100 may store labeling data including at least one of head coordinates, coordinates of a pupil center, and information related to camera settings according to an image in which a human face is captured. The eye coordinate learner 120 may train the second artificial neural network model using the data.

In addition, the eye coordinate learner 120 may train the second artificial neural network model using at least one of training data using the eye area image, training data obtained according to parameter changes of the virtual object, and labeling data.

According to an exemplary embodiment of the present specification, the third artificial neural network model may extract an eye area image, which is an image of an area including an eye extracted from each frame image of a video in which a human face is captured, by the eye rotation learner 130. The eye rotation learner 130 may train the third artificial neural network model to generate an eye rotation value using training data including information related to the eye phase changes generated according to the time sequence of the eye area image.

The eye rotation learner 130 may extract the eye area image from each frame image of the video. The eye rotation learner 130 may compare an eye area image extracted from an arbitrary frame image with eye area images extracted from frame images within a preset time range based on the corresponding frame image to generate the information related to the eye phase changes. For example, the eye rotation learner 130 may compare an eye area image extracted from an arbitrary frame image with eye area images extracted from frame images acquired within 0.1 seconds or so based on the corresponding frame image to generate the information related to the eye phase changes. This corresponds to an example and the present disclosure is not limited to the time.

According to an exemplary embodiment of the present specification, the eye rotation learner 130 may extract an iris area image, which is an image of an area occupied by an iris, from the eye area image. The iris area image may refer to an image inside a bounding box including an iris in the eye area image. The eye rotation learner 130 may train the third artificial neural network model using information related to a phase change of the iris according to the time sequence of the iris area image.

More specifically, the eye rotation learner 130 may compare an iris area image extracted from an arbitrary frame image with an iris area image extracted from frame images within a preset time range based on an arbitrary frame image. For example, the iris area image may be a mask image in which an area occupied by an iris is distinguished by different pixel values from other areas, which is an example, and the present disclosure is not limited thereto.

In addition, the eye rotation learner 130 may generate the information related to the phase change using the mask image of the iris generated by the eye coordinate learner 120.

The eye rotation learner 130 may generate the information related to the phase change of the iris by comparing the pixel values of the iris area image extracted from the arbitrary frame image with those of other iris area images. The eye rotation learner 130 may generate phase cross correlation values for pixel values of the iris area image extracted from the arbitrary frame image and other iris area images by using phase cross correlation analysis. The information related to the phase change calculated by using the phase cross correlation analysis may include contents about the change in the angle of the iris.

The eye rotation learner 130 may generate the phase cross correlation value by a method of obtaining a cross correlation value upsampled by a fast Fourier transform (FFT). The eye rotation learner 130 may calculate an initial estimate value of a cross correlation peak using the FFT, and then generate the phase cross correlation value by precisely estimating a phase shift of the upsampled signal using the discrete Fourier transform (DFT) in a preset area based on the estimated value. This corresponds to an example and the present disclosure is not limited to the method.

The eye rotation learner 130 may train the third artificial neural network model using the image and the phase cross correlation value of the iris area as the training data.

According to an exemplary embodiment of the present specification, the eye rotation learner 130 may calculate the size of the area occupied by the pupil in the eye area image extracted from the frame image of the m-th video. The eye rotation learner 130 may adjust the size of the target eye area image according to the preset criteria. The target eye area image refers to an eye area image extracted from the arbitrary frame image, and is not a term referring to a specific eye area image.

The eye rotation learner 130 may compare the sizes of the areas occupied by the pupils calculated from the target eye area image extracted from the arbitrary frame image and the preceding eye area image extracted from the immediately preceding frame image. The eye rotation learner 130 may adjust the size of the target eye area image so that the size of the area occupied by the pupil extracted from the target eye area image has a value within a preset difference value from the size of the area occupied by the pupil extracted from the preceding target eye area image. The eye rotation learner 130 may calculate the phase cross correlation value after adjusting the sizes of each eye area image.

In addition, the eye rotation learner 130 may generate a bounding box of an area including an eye in the frame image. The eye rotation learner 130 may extract the pupil center within the bounding box. Alternatively, the eye rotation learner 130 may receive the information related to the coordinates of the pupil center generated from the second artificial neural network model.

The eye rotation learner 130 may adjust the bounding box so that the pupil center is positioned at the center of the bounding box. The eye rotation learner 130 may extract an image inside the adjusted bounding box as the eye area image. The eye rotation learner 130 may extract the iris area image after adjusting the size of the eye area image according to the method described above and calculate the phase cross correlation value.

The eye rotation learner 130 may train the third artificial neural network model to generate the eye rotation value using the iris area image and the phase cross correlation value. The eye rotation value may refer to an angle of rotation clockwise or counterclockwise based on the central axis of the eye.

The eye rotation learner 130 may train the third artificial neural network model to generate the information related to the phase changes of the left and right eyes by extracting the eye area images for the left and right eyes from the frame image.

According to an exemplary embodiment of the present specification, the eye coordinate learner 120 may train the second artificial neural network model using the information generated from the first artificial neural network model. In addition, the eye rotation learner 130 may train the third artificial neural network model using the information generated from the second artificial neural network model.

For example, the eye coordinate learner 120 may generate the training data using the multiple frame images input to the first artificial neural network model and the information related to the head coordinates extracted from each frame image. The eye coordinate learner 120 may generate the eye area images from each frame image using each frame image and the information related to the head coordinates extracted from each frame image. The eye coordinate learner 120 may generate the eye area images from each frame image using the information related to the eye coordinates from the information related to the head coordinates. The eye coordinate learner 120 may train the second artificial neural network model according to the process described above.

The eye rotation learner 130 may train the third artificial neural network model further using the information related to the coordinates of the pupil center according to each frame image generated from the second artificial neural network model. In addition, the eye rotation learner 130 may train the third artificial neural network model by generating the training data according to the process described above using the image of the iris and/or pupil segmented by the eye coordinate learner 120.

The first to third artificial neural network models may be trained independently from each other, and may also be trained using the information generated from each artificial neural network model.

Hereinafter, the process of the balance function management system 10 according to the first exemplary embodiment generating the information on the balance function status and performing the balance function rehabilitation program using the trained artificial neural network model will be described.

The balance function management system 10 may acquire frame images of n videos in real time from n cameras that capture a subject performing a balance function status check and/or a balance function rehabilitation program.

When capturing the subject with one camera, the camera may be set to a value greater than a preset frames per second (FPS). For example, one camera may capture the subject at 240 FPS, which is an example, and the present disclosure is not limited to the value.

When capturing the subject with a plurality of cameras, the balance function management system 10 may control the sync of the plurality of cameras through at least one processor. For example, the at least one processor may control the sync of the plurality of cameras in real time using a technology such as Genlock, which is an example, and the present disclosure may control the sync of the plurality of cameras through a technology widely known to those skilled in the art.

In addition, the balance function management system 10 may sample frame images of the plurality of cameras through at least one processor. For example, one camera may capture the subject at 100 FPS, and another camera may capture the subject at 50 FPS. In this case, the balance function management system 10 may down-sample a video captured at 100 FPS by ½ times or up-sample a video captured at 50 FPS by 2 times using at least one processor. This is an example, and the present disclosure is not limited thereto.

Preferably, the subject may be captured by a plurality of cameras having the same frame rate.

The balance function management system 10 may acquire at least one of the head coordinates, the coordinates of the pupil center, and the information related to the eye phase changes of the subject using at least one of the first to third artificial neural network models.

The balance function management system 10 may acquire the head coordinates, the coordinates of the pupil center, and/or the information related to the eye phase changes using the first to third artificial neural network models.

In addition, the balance function management system 10 may generate the information related to the head coordinates, the coordinates of the pupil center, and the eye phase changes using an algorithm in which at least one processor generates the training data of the first to third artificial neural network models described above.

Hereinafter, it will be described that the information related to the head coordinates, the coordinates of the pupil center, and the eye phase changes is generated by using the first to third artificial neural network models. However, it is not necessary to use the artificial neural network model to generate the information.

The head coordinate acquirer 140 executes the first artificial neural network model stored in the memory 100, and inputs the frame image of the m-th video to the first artificial neural network model to acquire the information related to the head coordinates according to the m-th video. The information related to the head coordinates according to the m-th video may refer to the information related to the head coordinates generated according to the order of the frame images of the m-th video.

For example, when capturing the subject with two cameras, the head coordinate acquirer 140 may acquire the information related to the head coordinates according to the order of the frame images of the first video and the information related to the head coordinates according to the order of the frame images of the second video, which is an example, and the present disclosure is not limited to the number of cameras and videos.

When capturing the subject with a plurality of cameras, the head coordinate acquirer 140 may independently input the frame images of the m-th video to the first artificial neural network model. Alternatively, the head coordinate acquirer 140 may sequentially input the frame images of the m-th video to the first artificial neural network model.

For example, when capturing the subject with two cameras, the head coordinate acquirer 140 may input the frame image of the first video to the first artificial neural network model. Thereafter, the head coordinate acquirer 140 may input the frame image of the second video to the first artificial neural network model.

Alternatively, the head coordinate acquirer 140 may input the frame image of the second video to the first artificial neural network model and then input the frame image of the first video to the first artificial neural network model.

The head coordinate acquirer 140 may sequentially input the frame images of the m-th video to the first artificial neural network model according to a predetermined order. For example, the head coordinate acquirer 140 may input a first frame image of the first video to the first artificial neural network model and a first frame image of the second video to the first artificial neural network model. Thereafter, the head coordinate acquirer 140 may input a second frame image of the first video to the first artificial neural network model and a second frame image of the second video to the first artificial neural network model, which is an example, and the present disclosure is not limited to the order. In this case, the frame image input to the first artificial neural network model may include information on the extracted video.

The eye coordinate acquirer 150 may execute the second artificial neural network model stored in the memory 100 and input the information related to the head coordinates to the second artificial neural network model to acquire the information related to the coordinates of the pupil center according to the m-th video. The information related to the coordinates of the pupil center according to the m-th video may refer to the information related to the coordinates of the pupil center generated according to the order of the frame images of the m-th video.

For example, when capturing the subject with two cameras, the eye coordinate acquirer 150 may acquire the information related to the coordinates of a pupil center according to the order of the frame images of the first video and the information related to the coordinates of a pupil center according to the order of the frame images of the second video, which is an example, and the present disclosure is not limited to the number of cameras and videos.

The phase change acquirer 160 executes the third artificial neural network model stored in the memory 100 and inputs the information related to the coordinates of the pupil center according to the time sequence of the frame images of the m-th video to the third artificial neural network model to acquire the information related to the eye phase changes according to the m-th video. The information related to the eye phase changes according to the m-th video may refer to the information related to eye phase changes generated according to the order of the frame images of the m-th video.

For example, when capturing the subject with two cameras, the phase change acquirer 160 may acquire the information related to the eye phase changes according to the order of the frame images of the first video and the information related to the eye phase changes according to the order of the frame images of the second video, which is an example, and the present disclosure is not limited to the number of cameras and videos.

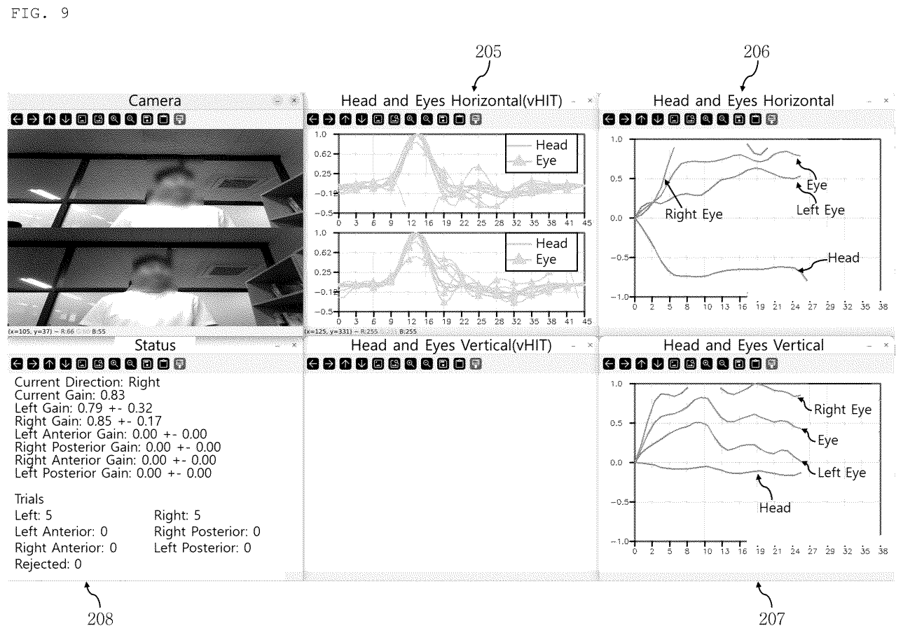

FIG. 8 is a block diagram of a balance function management system for generating information on a balance function status according to an exemplary embodiment of the present specification.

Referring to FIG. 8, a balance function management system 10-1 for generating balance function status information according to an exemplary embodiment the of present specification may include the memory 100, the head coordinate learner 110, the eye coordinate learner 120, the eye rotation learner 130, the head coordinate acquirer 140, the eye coordinate acquirer 150, the phase change acquirer 160, a head movement generator 1100, an eye movement generator 1110, a speed information generator 1120, and a balance function status information generator 1130.

Since the memory 100, the head coordinate learner 110, the eye coordinate learner 120, the eye rotation learner 130, the head coordinate acquirer 140, the eye coordinate acquirer 150, and the phase change acquirer 160 have been described above, a repetitive description thereof will be omitted.

The head movement generator 1100 may generate the information related to the head movement in the m-th video using the information related to the head coordinates acquired from the first artificial neural network model. The information related to the head movement may include horizontal movement and vertical movement of a head, and a degree of rotation of a head over time. The degree of rotation of the head may refer to a rotation angle of a head in the roll, pitch, and yaw directions. The information related to the head movement may be expressed as a graph of horizontal coordinate values and vertical coordinate values of the head over time.

According to an exemplary embodiment of the present specification, the head movement generator 1100 may calculate a normal vector of a subject's head using the feature points of the head generated in each frame image and the coordinates of the feature points. The direction of the normal vector may refer to the direction in which the front of the subject's head faces. The direction in which the front of the subject's head faces may refer to the direction in which the tip of the nose faces. The head movement generator 1100 may calculate a normal vector of the head using the feature points of the head, based on the feature point of the tip of the nose among the feature points of the head.

Alternatively, the direction in which the front of the subject's head faces may refer to the direction in which any feature point (such as the tip of the forehead or the center of the lips) that is on a straight line vertically based on the feature point of the tip of the nose faces. The head movement generator 1100 may calculate the normal vector of the head based on any one of the feature points.

Since calculating the normal vector for the front of the head using the feature point is a widely known technique among those skilled in the art, a detailed description thereof will be omitted.

The head movement generator 1100 may generate the information related to the head movement over time using the 3D head coordinate information and the normal vector according to the frame image of the m-th video. The head movement generator 1100 may output the information related to the head movement as a graph.

The eye movement generator 1110 may generate the information related to the eye movement in the m-th video using the information related to the coordinates of the pupil center and the eye phase changes generated from the second artificial neural network model and the third artificial neural network model. The information related to the eye movement may include the information related to the movement of the pupil center in the vertical direction, the movement of the pupil center in the horizontal movement, and the eye rotation value over time. The information related to the eye movement may be expressed as a graph of vertical coordinate value, horizontal coordinate value, and rotation angle of the pupil center of left and right eyes over time.

The eye movement generator 1110 may calculate a gaze vector of an eye using the vertical coordinate value, horizontal coordinate value, and rotation value of the pupil center. Since calculating the gaze vector using the coordinate value and rotation value of the pupil center is a widely known technique among those skilled in the art, a detailed description thereof will be omitted.

The eye movement generator 1110 may output the information related to the eye movement as a graph.

According to an exemplary embodiment of the present specification, the head movement generator 1100 and the eye movement generator 1110 may correct errors between the head movement and the eye movement information according to the training data and the head movement and the eye movement information of the subject.

The head movement and the eye movement information according to the training data may refer to actual data values for training the artificial neural network model.

For example, the actual data values may refer to parameter values of the Euler angles of the head and eyes acquired by the eye coordinate learner 120. The eye coordinate learner 120 may set parameter values of the virtual camera to be similar to actual settings in the balance function status check and/or the balance function rehabilitation program. The eye coordinate learner 120 may change parameter values of the Euler angle to be similar to head and eye movements of the subject according to the balance function status check and/or the balance function rehabilitation program. In this case, the head and eye movement information of the virtual object according to the change in the parameter values of the head and eyes may refer to actual data values. This is an example, and the actual data value may refer to the head and eye movement information according to the actual data value that may be used to train the first to third artificial neural network models.

The head movement generator 1100 and the eye movement generator 1110 may correct the errors in the head movement and the eye movement between the frame images acquired within a preset time. For example, when the preset time is 1.5 seconds and the camera captures the subject at 100 FPS, the head movement generator 1100 and the eye movement generator 1110 may correct the errors in the head movement and the eye movement between 150 frame images. This is an example, and the present disclosure is not limited to the time and frame rate.

The head movement generator 1100 and the eye movement generator 1110 may correct the errors of the head movement and the eye movement to have values within a preset range.

For example, the head movement generator 1100 and the eye movement generator 1110 may generate the information related to the head movement and the eye movement using the information related to the head coordinates, the coordinates of the pupil center, and/or the eye phase changes acquired from 150 frame images (frame images acquired for 1.5 seconds based on an arbitrary frame image) in the video in which the camera captures the subject at 100 FPS. The information related to the head movement and the eye movement may be calculated as the amount of head and eye movement (vertical, horizontal, rotation) over time according to the order of the frame images. In this case, the amount of head and eye movement at the time corresponding to the 50th frame image (frame image acquired 0.5 seconds after an arbitrary frame image) may be outside the preset error range. In this case, the head movement generator 1100 and the eye movement generator 1110 may calculate statistical values, such as the average or median of the amount of head and eye movement generated using information from the preceding frame image to replace the amount of movement at the time corresponding to the 50th frame image. Although it is described that the error is corrected between frame images acquired for 1.5 seconds based on an arbitrary frame image, this is an example, and various exemplary embodiments, such as frame images acquired 1.5 seconds before the arbitrary frame image and frame images acquired within 1.5 seconds or so, may occur, and this is an example, and the present disclosure is not limited to the time, frame rate, statistical values, etc.

As another example, the head movement generator 1100 and the eye movement generator 1110 may correct the errors of the head and eye movements by applying a filter. For example, the head movement generator 1100 and the eye movement generator 1110 may correct the errors using filters such as a chaining Kalman filter, a moving average filter, a Savitzky-Golay filter, a high pass filter, a low pass filter, and a band pass filter, which is an example, and the present disclosure is not limited thereto, and various types of filters may be used.

The speed information generator 1120 may generate the information related to the head and eye movement speeds in the m-th video using the information related to the head movement and the eye movement. The speed information generator 1120 may calculate the vertical speed of the head, the horizontal movement speed of the head, and/or the rotation speed of the head over time using the information related to the head movement. The speed information generator 1120 may calculate the vertical movement speed of the eye, the horizontal movement speed of the eye, and/or the rotation speed of the eye over time using the information related to the eye movement.

According to an exemplary embodiment of the present specification, the speed information generator 1120 may filter out noise values from the information related to the head and eye movement speeds. For example, when performing the balance function status check, noise values may occur in which the speeds of the head and eye movements are not accurately calculated by the case where the subject's eyes are covered, the case where the subject rotates his head quickly or slowly, or the case where the head position changes, etc. The speed information generator 1120 may remove noise from the information related to the head and eye movement speeds using the filters such as a chaining Kalman filter, a moving average filter, a Savitzky-Golay filter, a high pass filter, a low pass filter, or a band pass filter, and this is only an example, and the present disclosure is not limited thereto, and various noise processing methods may be used.

According to an exemplary embodiment of the present specification, the speed information generator 1120 may generate the information related to the head movement speed and the eye movement speed within a preset time based on a time when the head movement becomes greater than or equal to a preset threshold value. In the balance function status check, the subject may move the head in a horizontal (lateral left, lateral right) direction. In this case, the speed information generator 1120 may generate the information related to the head and eye movement speeds when the movement of the head in the horizontal direction is greater than or equal to the preset threshold value.

In addition, the subject may move the head in the lower right and upper right directions while the right side of the face is turned toward the front of the subject so that a right anterior semicircular canal and a left posterior semicircular canal (right anterior, left posterior (RALP)) are stimulated. In addition, the subject may move the head in the left-down and left-up directions while the left side of the face is turned toward the front of the subject so that the right posterior semicircular canal and the left anterior semicircular canal (left anterior, right posterior (LARP)) are stimulated. In this case, the speed information generator 1120 may generate the information related to the head and eye movement speeds when the movement of the head in the vertical direction is greater than or equal to the preset threshold value.

Hereinafter, the direction in which the subject rotates the head so that the RALP is stimulated will be described as the RALP direction, and the direction in which the subject rotates the head so that the LARP is stimulated will be described as the LARP direction.

For example, the speed information generator 1120 may determine whether the head movement is greater than or equal to a threshold value by using the head movement information according to a frame image existing within a preset time based on the last input frame image. The speed information generator 1120 may calculate the difference between the maximum and minimum values of the coordinates of the feature points of the head in the head movement information according to the frame image existing within the preset time to determine whether the head movement is greater than or equal to the threshold value. The preset threshold value may vary depending on the frame rate of the video, the size of the frame image, etc.

As another example, the memory 100 may further store a fourth artificial neural network model that generates the information on the head movement. The fourth artificial neural network model may be trained by allowing at least one processor to use the frame image of the video performing the balance function status check and the data of the pitch and yaw values of the head in the corresponding frame image. The fourth artificial neural network model may be a time series model or a transformer model, which is an example, and the present disclosure is not limited to the model. In this case, the frame image may be labeled with information on the lateral, RALP, and LARP directions. The speed information generator 1120 may input a frame image existing within a preset time based on the last acquired frame image to the fourth artificial neural network model to confirm whether the head movement is greater than or equal to the threshold value.

The speed information generator 1120 may calculate the head and eye movement speeds using the information related to the head and eye movement generated during a preset time after the time when the head movement becomes greater than or equal to the threshold value. For example, the speed information generator 1120 may calculate the head and eye movement speeds using the information related to the head and eye movement generated within 1.5 seconds after the time when the head movement becomes greater than or equal to the threshold value, which is an example, and the present disclosure is not limited to the time. The speed information generator 1120 may calculate the head and eye movement speeds in the m-th video, respectively.

The speed information generator 1120 may display the information related to the head and eye movement speeds on the display device. The speed information generator 1120 may display the information related to the head and eye movement speeds from which noise has been removed and/or the information related to the head and eye movement speeds from which noise has not been removed on the display.

The balance function status information generator 1130 may generate the information on the balance function status of the subject using the information related to the head and eye movement speeds.

According to an exemplary embodiment of the present specification, the balance function status information generator 1130 may calculate a gain coefficient using a time value (head peak index) when the head movement speed of the subject is relatively the largest within a preset analysis window and a time value (eye peak index) when the eye movement speed is relatively the largest when the eye of the subject moves in the direction of the head movement and then returns to the original position. The analysis window may mean a preset time range based on the time when the head movement becomes greater than or equal to the threshold value. The size of the analysis window may correspond to a time range in which the speed information generator 1120 generates the information related to the head and eye movement speeds.

For example, the size of the analysis window may be 1.5 seconds after the time when the head movement becomes greater than or equal to the threshold value, which is an example, and the present disclosure is not limited thereto.

The balance function status information generator 1130 may calculate the gain coefficient using [Equation 1] using the head peak index and the eye peak index from the information related to the head and eye movement speeds from which noise has been removed.

$$Gain Coefficient = \frac{Window Size - \vert Phase Shift \vert}{Window Size} \qquad [ Equation 1 ]$$

$$Window Size : Size ( time ) of analysis window$$

$$Phase Shift : Difference between Head Peak Index and Eye Peak Index$$

Thereafter, the balance function status information generator 1130 may calculate gain using the gain coefficient.

$$Gain = \frac{Eye Extrema}{Head Extrema} \begin{matrix} \\ \\\end{matrix} Gain Coefficient \qquad [ Equation 2 ]$$

$$Eye Extrema : Maximum Speed of Pupil$$

$$Head Extrema : Maximum Speed of Head$$

The balance function status information generator 1130 may calculate the gains for the left eye and the right eye, respectively.

In addition, the balance function status information generator 1130 may calculate the gains in the m-th video, respectively. For example, when the subject is captured using two cameras, the gains for the two videos may be calculated, respectively. In this case, the gains of the left eye and the right eye in the first video and the gains of the left eye and the right eye in the second video may be calculated. The balance function status information generator 1130 may calculate a statistical value for the gain of the left eye calculated in the first video and the second video, and calculate at least one statistical value for the gain of the right eye calculated in the first video and the second video. The statistical value may correspond to an average, a median, a minimum, a maximum, a standard deviation, etc., and this is an example, and the present disclosure is not limited thereto.

According to an exemplary embodiment of the present specification, when the subject is captured by a plurality of cameras (n is 2 or more), the head movement generator 1100 may further generate reference head movement information by calculating the statistical values of the information related to the head coordinates according to the m-th video. The eye movement generator 1110 may further generate the reference eye movement information by calculating the statistical values of the information related to the coordinates of a pupil center and the eye phase changes according to the m-th video.

When the subject is captured by the plurality of cameras, a difference in the coordinate values of the 3D head generated from the frame images of the m-th video may occur depending on the position, angle, etc., of the cameras.

For example, a first camera may capture the subject from the right side of the subject, and a second camera may capture the subject from the left side of the subject. In this case, when the subject turns his/her head to the right, the coordinates of the feature points positioned on the right side of the face of the subject may be calculated more accurately than the coordinates of the feature points positioned on the left side in the frame image of the first video captured by the first camera. In addition, when the subject turns his/her head to the left, the coordinates of the feature points positioned on the left side of the face of the subject may be calculated relatively more accurately than the coordinates of the feature points positioned on the right side in the frame image of the second video captured by the second camera.

As another example, a plurality of cameras may be installed to surround the subject at 15° intervals at a distance of 1 m from the subject. In this case, the plurality of cameras may capture the subject at an eye height of the subject. Even in this case, the coordinates of the feature points that are measured relatively more accurately for each camera may be generated depending on the direction of the head movement of the subject. In this way, the information of the two-dimensional head coordinates generated depending on the positions, angles, etc., of the plurality of cameras may be different from each other.

The head movement generator 1100 may generate the reference head coordinate information by calculating the average value of the coordinates of each feature point in the three-dimensional head coordinates generated from the frame images that are synchronized with each other in the m-th video. The head movement generator 1100 may further generate the information related to the reference head movement using the reference head coordinate information over time.

The eye movement generator 1110 may generate the information related to the reference coordinates of the pupil center and the reference eye phase change by calculating an average value of the information related to the coordinates of the pupil center and the eye phase change generated from the frame images that are synchronized in the m-th video. The eye movement generator 1110 may generate a reference gaze vector of the eye using the information related to the reference coordinates of the pupil center and the reference eye phase change. The eye movement generator 1110 may further generate the information related to the reference eye movement using the reference coordinates of the pupil center, the information related to the reference eye phase change, and the reference gaze vector.

In this case, the speed information generator 1120 may generate the information related to the head movement speed and the eye movement speed for the m-th video, respectively, within a preset time based on the time when the reference head movement becomes greater than or equal to the preset threshold value.

The balance function status information generator 1130 may calculate the gains using the head peak index and the eye peak index for the m-th video within the preset time based on the time when the reference head movement becomes greater than or equal to the preset threshold value.

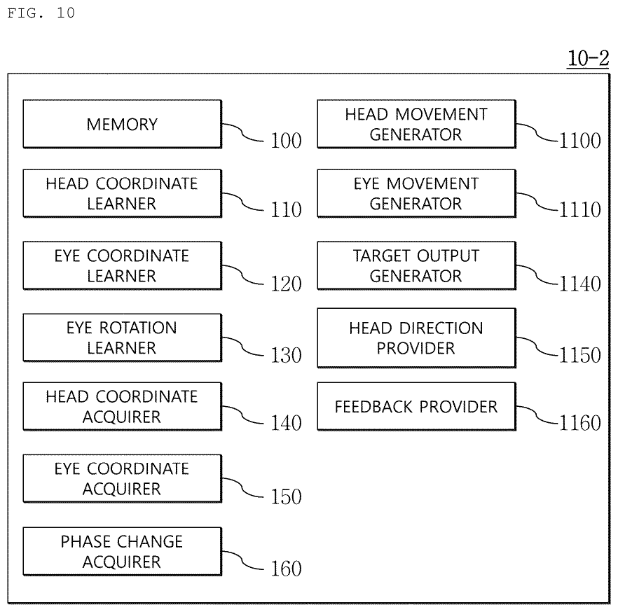

FIG. 9 is a diagram illustrating an example of outputting information on a balance function status to a display according to an exemplary embodiment of the present specification.

Referring to FIG. 9, the head movement generator 1100, the eye movement generator 1110, the speed information generator 1120, and the balance function status information generator 1130 may output the calculated information on the display screen. On the display screen, videos captured by the plurality of cameras may be outputted, respectively.

The speed information generator 1120 may output the information on the head and eye movement speeds for the m-th video as a graph 205, respectively. In the graph 205 of the head and eye movement speeds, the speed information according to the number of times of balance function status checks may be superimposed and displayed. In the speed graph 205 of the head and eye movements, the time values at which a peak and/or valley appear may correspond to the head peak index and/or the eye peak index. In the graph, the vertical axis may correspond to the speed value, and the horizontal axis may correspond to the time value. Although the graph for the horizontal speed of the head and eye is illustrated in FIG. 9, this is an example, and graphs for the speed in the vertical and rotation directions may be further outputted depending on the type of test.

The head movement generator 1100 and the eye movement generator 1110 may output the head and eye movement information as graphs. The head movement generator 1100 and the eye movement generator 1110 may output a movement graph 206 of the head and eye in a horizontal direction and a movement graph 207 of the head and eye in a vertical direction. In addition, the movement graph for the rotation direction of the head and/or eye may be further output. In addition, the head and eye movement graphs may include the movement information for the m-th video, and may include the reference head movement and reference eye movement information.

The balance function status information generator 1130 may output information 208 related to the balance function status check for the subject to a display device. The information related to the balance function status check may include at least one of the rotation direction (lateral, RALP, LARP) of the head, a gain and standard deviation according to the head rotation direction, the number of balance function status checks according to the rotation direction of the head, the number of times of successful calculations of the gain, and the number of times of failures in the calculation of the gain.

The case where the calculation of the gain fails may occur when the subject moves the head faster than the standard of the test. In this case, the difference between the head peak index and the eye peak index may exceed the middle of the analysis window size. In this case, the balance function status information generator 1130 may fail to calculate the gain. The balance function status information generator 1130 may generate the information on the number of times of successful and failed calculations of the gain, thereby providing an effect in which the subject and/or the examiner may make a more accurate determination.

In addition, the balance function status information generator 1130 may generate information on whether the semicircular canal is abnormal according to the gain. For example, the balance function status information generator 1130 may generate information on the abnormality of the semicircular canal when the gains of the left and right eyes are lower than or equal to the preset value in the balance function status check.

In addition, the subject may rotate his/her head in the lateral direction in the balance function status check. In this case, when the difference in the gains of the left and right eyes when the subject turns his/her head to the left and to the right is greater than or equal to the preset value, the balance function status information generator 1130 may generate abnormal information of the semicircular canal.

In addition, in the balance function status check, the subject may rotate his/her head in the RALP or LARP direction. In this case, when the difference in the gains of the left and right eyes when the subject turns his/her head upward and downward is greater than or equal to the preset value, the balance function status information generator 1130 may generate abnormal information of the RALP or LARP.

In addition, the balance function status information generator 1130 may further generate the information on whether there is an abnormality in the central vestibular nerve function and an abnormality in the peripheral vestibular nerve function by using the information related to the eye movement and the gain information.

FIG. 10 is a block diagram of a balance function management system for performing a balance function rehabilitation program according to an exemplary embodiment of the present specification.

Referring to FIG. 10, a balance function management system 10-2 for performing a balance function rehabilitation program according to an exemplary embodiment of the present specification may include the memory 100, the head coordinate learner 110, the eye coordinate learner 120, the eye rotation learner 130, the head coordinate acquirer 140, the eye coordinate acquirer 150, the phase change acquirer 160, the head movement generator 1100, the eye movement generator 1110, a target output generator 1140, a head direction provider 1150, and a feedback provider 1160. Since the memory 100, the head coordinate learner 110, the eye coordinate learner 120, the eye rotation learner 130, the head coordinate acquirer 140, the eye coordinate acquirer 150, the phase change acquirer 160, the head movement generator 1100, and the eye movement generator 1110 have been described above, a repetitive description thereof will be omitted.

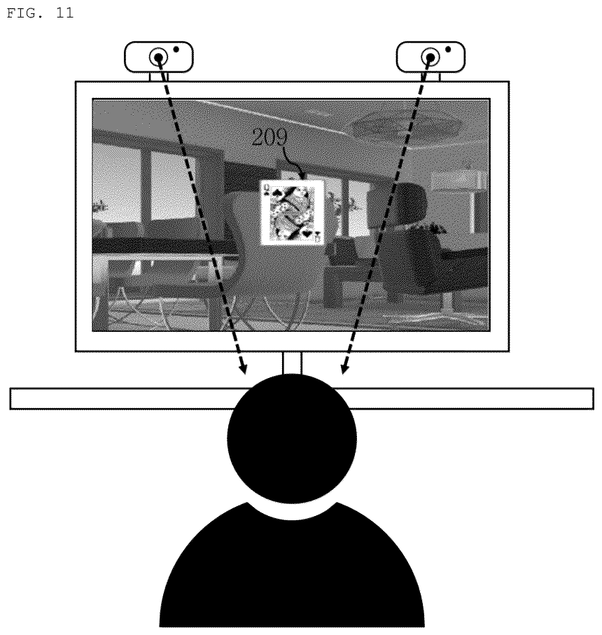

FIG. 11 is a diagram illustrating an example of a scene performing the balance function rehabilitation program according to an exemplary embodiment of the present specification.

Referring to FIG. 11, the target output generator 1140 may output a virtual target 209 to the display device. The virtual target may be displayed at any position on the display device. In FIG. 11, the target is illustrated in the shape of a trump card, but this is only an example, and the present disclosure is not limited to the shape.

The head direction provider 1150 may provide the subject with the information on the rotation direction of the head according to the balance rehabilitation protocol. For example, the head direction provider 1150 may provide information so that the subject rotates the head in the lateral direction.

Alternatively, the head direction provider 1150 may provide information so that the subject rotates the head in the RALP or LARP direction.

In this case, the head direction provider 1150 may provide information so that the subject rotates the head only upward or downward while the right side of the head is facing forward. In addition, the head direction provider 1150 may provide information so that the subject rotates the head only upward or downward while the left side of the head is facing forward.

In addition, the head direction provider 1150 may provide information so that the subject returns to a state before rotating the head within a preset time after rotating the head. For example, the head direction provider 1150 may provide information to return to a state before rotating the head after rotating the head within 1 second.

The head direction provider 1150 may visually display the information on the display device. In addition, the head direction provider 1150 may output the information to an audio device.

The feedback provider 1160 may provide feedback according to the head movement and the eye movement of the subject.

According to the balance rehabilitation protocol, the reference of the angle at which the subject should rotate the head may be determined in advance. The feedback provider 1160 may compare the rotation angle of the subject's head generated from the head movement generator 1100 with the reference of the angle. The feedback provider 1160 may provide auditory feedback and/or visual feedback when the rotation angle of the subject's head satisfies the reference of the angle. In addition, the feedback provider 1160 may provide auditory feedback and/or visual feedback when the rotation angle of the subject's head does not satisfy the reference of the angle. The feedback provider 1160 may provide different feedbacks depending on whether the rotation angle of the subject's head satisfies the reference of the angle.

In addition, the eye movement generator 1110 may generate the coordinate information of the gaze point which the subject's gaze faces on the display device using the eye gaze vector. The feedback provider 1160 may compare the coordinate information of the gaze point generated from the eye movement generator 1110 with the coordinate information of the virtual target 209.

When the coordinates of the gaze point are positioned within the area of the virtual target 209, the feedback provider 1160 may change the color value of the virtual target 209. In addition, the feedback provider 1160 may further display the gaze point within the virtual target 209.

When the coordinates of the gaze point are positioned outside the area of the virtual target 209, the feedback provider 1160 may display the position of the gaze point on the display device.

When the subject is captured by the plurality of cameras, the head movement generator 1100 and the eye movement generator 1110 may generate the information related to the reference head movement and the reference eye movement as described above. The feedback provider 1160 may provide the feedback according to the information related to the reference head movement and the reference eye movement.

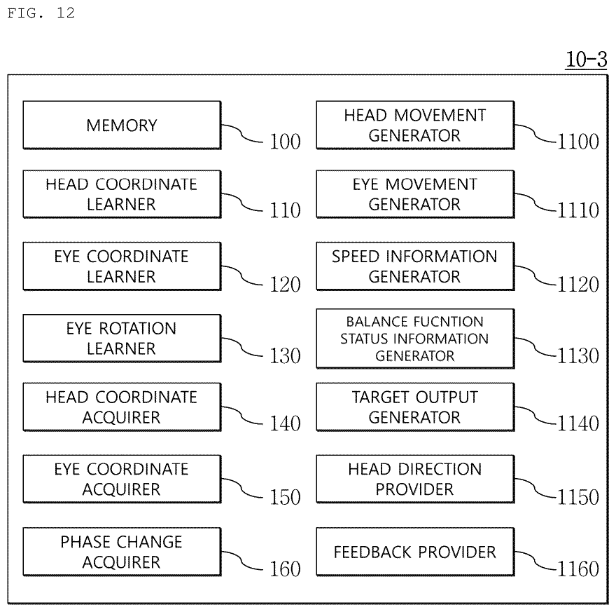

FIG. 12 is a block diagram of a balance function management system for generating information on a balance function status and performing a balance function rehabilitation program according to an exemplary embodiment of the present specification.

Referring to FIG. 12, the balance function management system 10-3 may include the memory 100, the head coordinate learner 110, the eye coordinate learner 120, the eye rotation learner 130, the head coordinate acquirer 140, the eye coordinate acquirer 150, the phase change acquirer 160, the head movement generator 1100, the eye movement generator 1110, the speed information generator 1120, the balance function status information generator 1130, the target output generator 1140, the head direction provider 1150, and the feedback provider 1160. The balance function management system 10-3 may generate a gain to provide a balance function rehabilitation program according to whether the balance function is abnormal.

Hereinafter, a balance function management system according to a second exemplary embodiment of the present specification will be described. According to the second exemplary embodiment of the present specification, the balance function management system may generate information on head and eye movements of a subject using multiple videos captured by a plurality of cameras to generate information on a balance function status and/or perform a balance function rehabilitation program. Hereinafter, n may mean a natural number greater than or equal to 2.

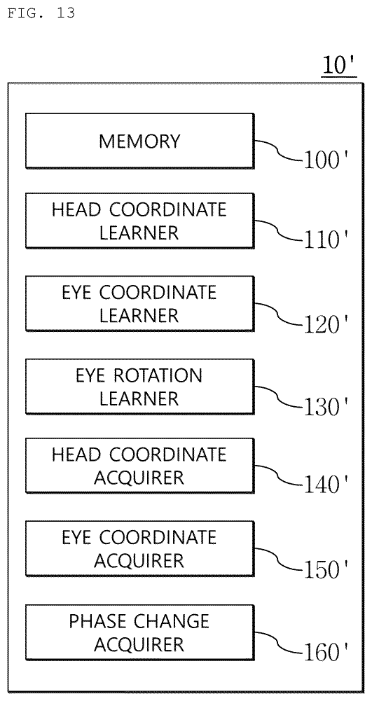

FIG. 13 is a block diagram of a balance function management system according to a second exemplary embodiment of the present specification.

Referring to FIG. 13, a balance function management system 10′ according to the second exemplary embodiment of the present specification may include a memory 100′, a head coordinate learner 110′, an eye coordinate learner 120′, an eye rotation learner 130′, a head coordinate acquirer 140′, an eye coordinate acquirer 150′, and a phase change acquirer 160′.

The memory 100′ may store at least one of a first artificial neural network model that generates information related to head coordinates, a second artificial neural network model that acquires information related to coordinates of a pupil center, and a third artificial neural network model that acquires information related to eye phase changes.

According to an exemplary embodiment of the present specification, the first artificial neural network model may be trained by allowing the head coordinate learner 110 to use facial feature points and coordinates of the feature points extracted from frame images of multiple videos in which a human face is captured as training data.

The head coordinate learner 110′ may train the first artificial neural network model using at least multiple videos in which a human face is captured. The multiple videos in which the human face is captured may be a video in which an appearance in which the captured human head rotates is captured.

For example, the video in which the human face is captured may be captured by a plurality of cameras. The plurality of cameras may be installed at various angles at various positions to capture a person. The video in which the human face is captured may be captured by cameras installed at various angles at various positions such as the left, right, upper left, and upper right of the front of the person. The video captured by the plurality of cameras may refer to a video in which the plurality of cameras captures the same situation.

The video in which the human face is captured may be a video in which a scene where a head moves left and right is captured while the captured human face faces forward. In addition, the video in which the human face is captured may be a video in which a scene where a head moves up and down is captured while the captured human face faces forward. The state in which the human face faces forward may be a state in which a gaze direction of a person and a front direction of a body are consistent.

In addition, the video in which the human face is captured may be a video in which the scene where the head moves up and down is captured after the captured human head is turned to the right. In addition, the video in which the human face is captured may be a video in which the scene where the head moves up and down is captured after the captured human head is turned to the left.

In addition, the video in which the human face is captured may be a video in which the scene where the head rotates is captured while the captured human gazes at a specific gaze point.

The video in which the human face is captured may be captured by cameras in which all the specifications of the cameras are the same. In addition, the video in which the human face is captured may be a video that is captured by cameras having different specifications. In addition, the video in which the human face is captured may be a video that is captured by cameras having different setting values.

The above-described video in which the person is captured corresponds to an example, and the video in which the human head and eye are captured or all the videos in which the human head is captured may be used. In addition, the present disclosure is not limited to specific camera specifications, settings, etc.

Preferably, the video in which the person is captured may be a video captured by a camera having the same setting value.

According to an exemplary embodiment of the present specification, the head coordinate learner 110′ may extract feature points after adjusting the sync of the multiple videos when extracting the feature points from the multiple videos in which a person is captured using a plurality of cameras. The head coordinate learner 110′ may adjust the sync of the multiple videos using an algorithm such as a specific audio signal of the multiple videos and feature point matching-based synchronization, which is an example, and the present disclosure is not limited thereto, and various technologies widely known to those skilled in the art may be used.

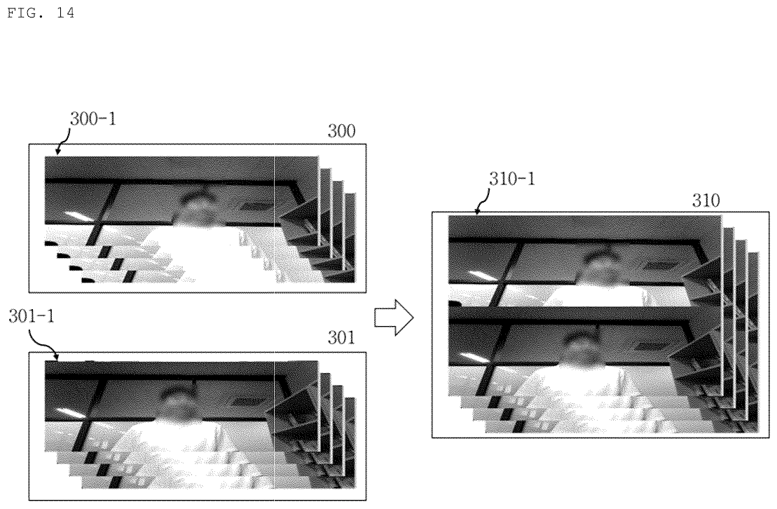

FIG. 14 is a reference diagram illustrating an example in which a head coordinate learner concatenates frame images according to an exemplary embodiment of the present specification.

Referring to FIG. 14, the video in which the human face is captured may be a video captured by two cameras. The head coordinate learner 110′ may adjust the sync of the first video 300 captured by the first camera and the second video 301 captured by the second camera. The head coordinate learner 110′ may generate a multi-frame image 310 by concatenating frame images having the same sync in the first video 300 and the second video 301. The head coordinate learner 110′ may generate a multi-frame image 310 by concatenating frame images having the same sync. For example, the first frame image 300-1 of the first video 300 and the first frame image 301-1 of the second video 301 may be concatenated to generate a first multi-frame image 310-1. The multi-frame image may refer to one image generated by concatenating multiple frame images having the same sync. The multi-frame image may refer to one image in which the multiple frame images are arranged.

Hereinafter, the multi-frame image will be described assuming that the frame images of the m-th video having the same sync among the frame images of n videos are concatenated.

The head coordinate learner 110′ may generate a multi-frame image by concatenating the frame images of the m-th video having the same sync among n videos captured by n cameras. The head coordinate learner 110′ may concatenate the frame images of the m-th video according to the preset criteria. In addition, the head coordinate learner 110′ may label information of the video from which the frame images are extracted.

The head coordinate learner 110′ may extract feature points for a human face from the multi-frame image. For example, the head coordinate learner 110′ may extract feature points positioned on a human face from each multi-frame image. For example, the feature points may include feature points for a tip of a nose, a left outer canthus (point where an outermost eyelid of a left eye meets), a right outer canthus (point where an outermost eyelid of a right eye meets), and a forehead of a person. The positions of the feature points may not change even if a person blinks. The feature points correspond to an example, and the present disclosure is not limited to the feature points, and feature points used for conventional face recognition may be further included. Since the technology for generating feature points and feature point coordinates from a human head is a technology widely known to those skilled in the art, a detailed description thereof will be omitted.

The head coordinate learner 110′ may generate coordinate information of feature points extracted from each frame image. The head coordinate learner 110′ may train the first artificial neural network model to generate 3D head coordinates for each multi-frame image using training data including feature points extracted from each multi-frame image and coordinates of the feature points. The 3D head coordinates may refer to 3D coordinates of a head according to a 3D standard head model. The 3D coordinates of the head may include coordinates for all feature points of the head. The 3D coordinates of the head may include contents related to an index number for each feature point. In this case, the head coordinate learner 110′ may train the first artificial neural network model to generate the index number based on the feature points of the tip of the nose.

The head coordinate learner 110′ may train the first artificial neural network model to generate the 3D coordinates of the head for the frame image of the m-th video in the multi-frame image. For example, when the multi-frame image is the multi-frame image which concatenates frame images of two videos, the head coordinate learner 110′ may train the first artificial neural network model to generate a three-dimensional coordinate of the head for the frame image of the first video and a three-dimensional coordinate of the head for the frame image of the second video, which is an example, and the present disclosure is not limited to the number of videos.

In addition, the head coordinate learner 110′ may train the first artificial neural network model to generate the reference head coordinate for the three-dimensional coordinates of the head. The reference head coordinate may refer to an average value of the coordinates of each feature point in the three-dimensional coordinates of the head generated from the frame image of the m-th video. The head coordinate learner 110′ may train the first artificial neural network model to generate the information related to the reference head coordinate in which the difference in the head coordinates is corrected due to the position and angle of the camera by calculating the average value.

In addition, the head coordinate learner 110′ may train the first artificial neural network model to further generate the head movement information using the 3D coordinates extracted from each multi-frame image.

In addition, the head coordinate learner 110′ may train the first artificial neural network model further using, as training data, data on a 3D standard head model, such as the frame image from which the feature points are extracted, a 3D morphable model (3DMM), a faces learned with an articulated model and expressions (FLAME) model, which is an example, and the present disclosure is not limited to the training data, and various training data may be additionally used.

According to an exemplary embodiment of the present specification, the head coordinate learner 110′ may use feature points having coordinate values according to preset criteria as training data. The head coordinate learner 110′ may select the feature points having the coordinate values according to the preset criteria as training data.

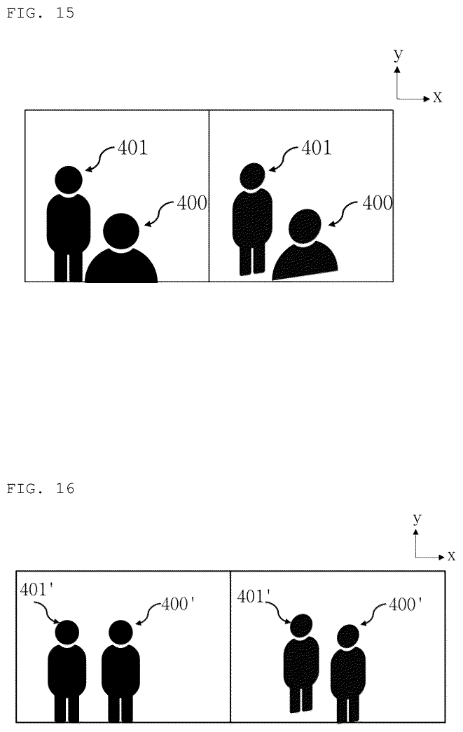

FIG. 15 is a diagram illustrating an example of a multi-frame image of a scene where a subject performing a balance function status check and/or a balance function rehabilitation program according to an exemplary embodiment of the present specification is captured.

Referring to FIG. 15, the multiple videos in which the person is captured may be multiple videos of performing the balance function status check and/or the balance function rehabilitation program using the balance function management system 10′. The video may be a video in which only a subject 400 is captured. In addition, the video may be a video in which the subject 400 and an examiner 401 are captured. Hereinafter, the video in which the person is captured will be described as meaning the video of performing the balance function status check and/or the balance function rehabilitation program. However, this corresponds to an example and the present disclosure is not limited to the video.

When performing the balance function status check, the subject 400 may be sitting on a chair and the examiner 401 may be standing behind the subject. The examiner 401 may refer to a medical professional. The balance function management system 10′ may analyze only the head movement and the eye movement of the subject 400 to determine whether the balance function of the subject is abnormal by analyzing the eye movement according to the head movement of the subject 400 and to proceed with the rehabilitation program.

The head coordinate learner 110′ may extract the facial feature points and the coordinates of the feature points of the subject 400 from the multi-frame image to train the first artificial neural network model. The head coordinate learner 110′ may extract the facial feature points and the coordinates of the feature points of the subject from the frame image of the m-th video from the multi-frame image to train the first artificial neural network model.

As described above, since the subject 400 is sitting on a chair and the examiner 401 is standing, the feature points extracted from the face of the subject 400 may be positioned relatively lower than the feature points extracted from the face of the examiner 401. This may refer to that a y-coordinate value of the feature points extracted from the face of the subject 400 is relatively smaller than a y-coordinate value of the feature points extracted from the face of the examiner 401.

The head coordinate learner 110′ may extract, as training data, feature points with relatively smaller y-coordinate values among feature points of the same portion extracted from the faces of the subject 400 and the examiner 401. The head coordinate learner 110′ may extract feature points positioned in an area below the preset y-coordinate value as training data.

In addition, the head coordinate learner 110′ may cluster feature points extracted from the faces of the subject 400 and the examiner 401. Thereafter, the head coordinate learner 110′ may extract a set of feature points positioned at a relatively lower side as training data.

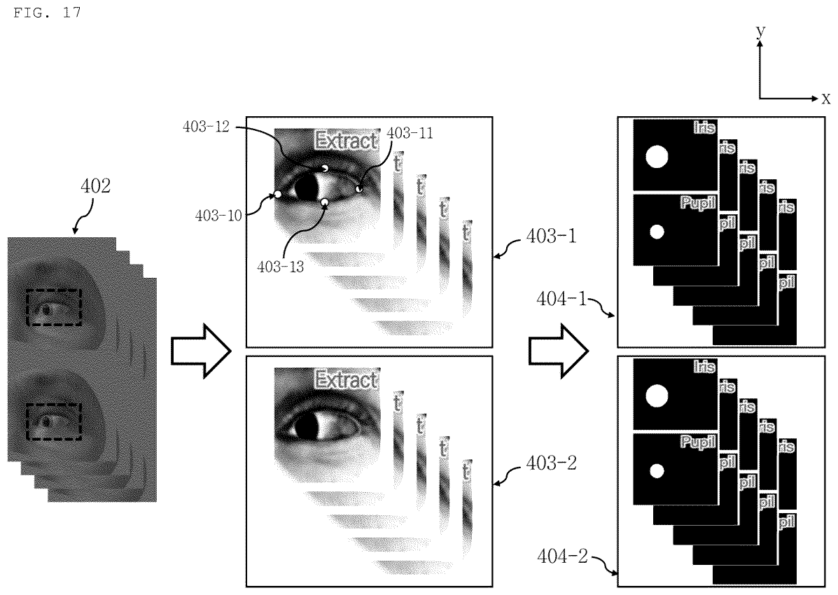

FIG. 16 is a diagram illustrating an example of the multi-frame image of the scene where the subject performing the balance function status check and/or the balance function rehabilitation program according to an exemplary embodiment of the present specification is captured.

Referring to FIG. 16, in the video, the balance function status check and/or the balance function rehabilitation program may be performed while both the subject 400′ and examiner 401′ are standing. In this case, the feature points extracted from the face of the subject 400′ may be relatively closer to the center of the display screen than the feature points extracted from the face of the examiner 401′. The head coordinate learner 110′ may extract the feature points having the coordinate values that are relatively closer to the center of the display screen as training data.

In another example, the subject may be relatively closer to the camera than the examiner. As a result, the face of the subject may occupy a relatively wider area than the face of the examiner in the video. The head coordinate learner 110′ may extract, as training data, a set of feature points which are distributed over a relatively wider area, among a set of feature points extracted from the face of the subject and a set of feature points extracted from the face of the examiner.

The head coordinate learner 110′ may generate the feature points and the coordinate information of the feature points from the face of the subject included in the frame image of the m-th video in the multi-frame image, respectively.

The process in which the head coordinate learner 110′ described above extracts only the feature points extracted from the face of the subject as training data is an example, and the present disclosure is not limited thereto, and various criteria may be set according to the position of the subject, the position of the examiner, the position of the camera, the angle, etc. In addition, only the feature points extracted from the face of the subject may be extracted as training data through markers, etc., for distinguishing the subject and the examiner as well as the positions of the faces of the subject and the examiner. Therefore, various exemplary embodiments may arise depending on various situations in which the balance function status check and/or the rehabilitation examination program are performed.

In the video, when the head of the subject quickly rotates, the face of the subject may not be clearly captured in at least one of the frame images of the m-th video. In this case, the head coordinate learner 110′ may not extract the feature points from the face of the subject. In this case, the head coordinate learner 110′ may train the first artificial neural network model using the feature points extracted from the face of the examiner, so the first artificial neural network model may produce inaccurate results.

According to an exemplary embodiment of the present specification, the head coordinate learner 110′ may track the coordinates of the feature points extracted from the frame image preceding an arbitrary frame image of the m-th video. As described above, when the feature points are not extracted from the face of the subject in the frame image of the m-th video, the head coordinate learner 110′ may extract the training data using the image of the subject in the preceding frame image. The preceding frame image may refer to the closest frame image from which feature points may be extracted from the face of the subject among the frame images preceding the arbitrary frame image.

According to an exemplary embodiment of the present specification, the second artificial neural network model may be trained to generate the information related to the coordinates of the pupil center by allowing the eye coordinate learner 120′ to use the training data including the eye area image, which is an image of an area including an eye in the multi-frame image of the multiple videos in which the human face is captured.

The eye coordinate learner 120′ may generate the multi-frame image by controlling the sync of the multiple videos. Alternatively, the eye coordinate learner 120′ may receive the multi-frame image generated from the head coordinate learner 110′.

The eye coordinate learner 120′ may extract an eye area image of a subject, which is an image of an area including an eye of the subject, from the multi-frame image. The eye coordinate learner 120′ may train the second artificial neural network model to generate the information related to the coordinates of the pupil center by using the eye area image of the subject extracted from each frame image as training data.

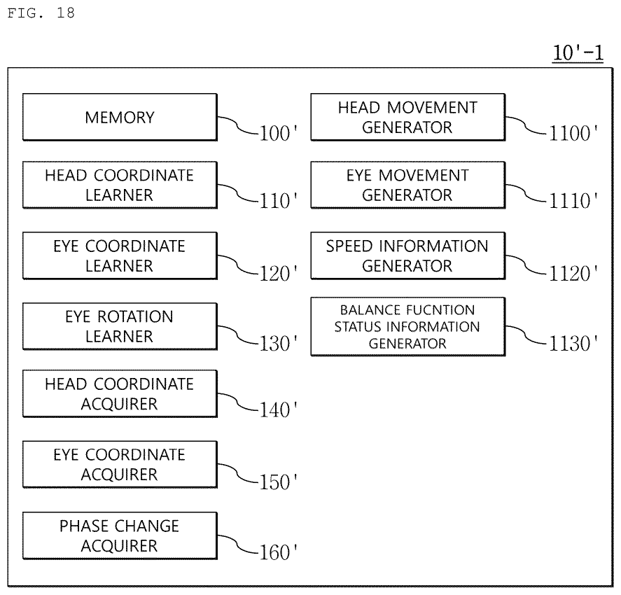

FIG. 17 is a diagram illustrating an example of preprocessing an eye area image according to an exemplary embodiment of the present specification.

Referring to FIG. 17, the eye coordinate learner 120′ may extract eye area images 403-1 and 403-2 according to the frame images of the m-th video from each multi-frame image 402. The eye coordinate learner 120′ may extract an image inside a bounding box of an eye area from each multi-frame image 402 as eye area images 403-1 and 403-2. The eye coordinate learner 120′ may segment the iris and pupil areas from the eye area images 403-1 and 403-2. Since the technology of extracting the eye area from the human face and segmenting the iris and pupil areas is a technology widely known to those skilled in the art, a detailed description thereof will be omitted.

The eye coordinate learner 120′ may estimate an area for a part where the iris and/or pupil are covered by an eyelid. As illustrated in FIG. 17, a part of the iris may be covered by an upper eyelid and a lower eyelid. The eye coordinate learner 120′ may estimate the covered part using an ellipse fitting algorithm, a circle Hough transform algorithm, or the like. Alternatively, the eye coordinate learner 120′ may segment the iris and pupil areas using the artificial neural network model that has been previously trained to segment the iris and pupil areas. This corresponds to an example and the present disclosure is not limited to the method.

The eye coordinate learner 120′ may train the second artificial neural network model using data in which the iris and/or pupil areas are segmented in the eye area images 403-1 and 403-2. For example, the eye coordinate learner 120′ may segment the iris and/or pupil areas from the eye area images 403-1 and 403-2 to generate mask images 404-1 and 404-2. The eye coordinate learner 120′ may generate a mask image in which the area occupied by the iris and/or pupil and the remaining area have different pixel values in the eye area images 403-1 and 403-2. The mask image may be displayed in white or black for the area occupied by the iris and/or pupil, and displayed in black or white for the remaining area, which is an example, and the present disclosure is not limited thereto.

The eye coordinate learner 120′ may train the second artificial neural network model to generate the information related to the coordinates of the pupil center for frame images of each video using the mask images 404-1 and 404-2 as the training data. Alternatively, the eye coordinate learner 120′ may train the second artificial neural network model using two-dimensional pixel values of the mask images 404-1 and 404-2 as training data. Alternatively, the eye coordinate learner 120′ may train the second artificial neural network model using the mask images 404-1 and 404-2 and the two-dimensional pixel values as the training data.

As another example, the eye coordinate learner 120′ may train the second artificial neural network model using a heatmap model that segments and displays the iris and/or pupil area in the eye area images 403-1 and 403-2.

The eye coordinate learner 120′ may train the second artificial neural network model using at least one of the mask image and the heatmap model.

In FIG. 17, a process of performing preprocessing using a multi-frame image in which frame images of two videos are concatenated is illustrated, but this is only an example, and the present disclosure is not limited to the number of videos.

According to an exemplary embodiment of the present specification, the eye coordinate learner 120′ may train the second artificial neural network model to generate the eye feature points and coordinate information of the feature points from the eye area images 403-1 and 403-2, and to generate the horizontal coordinate values and vertical coordinate values of the pupil center for the frame images of each video using the coordinates of the plurality of preset feature points. The eye coordinate learner 120′ may extract normalized coordinates of a pupil center using the coordinates of the plurality of feature points extracted from the eye area images 403-1 and 403-2.

For example, the coordinates of the plurality of feature points may include a feature point having a relatively smallest x-axis coordinate value and a feature point having a relatively largest x-axis coordinate value among feature points whose y-axis coordinates are within a preset range in the eye area image 403-1.

The coordinates of the plurality of feature points may include a feature point having a relatively smallest y-axis coordinate value and a feature point having a relatively largest y-axis coordinate value among feature points whose x-axis coordinates are within a preset range in the eye area image 403-1 captured from the front of the person.

For example, the eye coordinate learner 120′ may extract horizontal coordinates of a normalized pupil center using a feature point 403-10 for a medial canthus and a feature point 403-11 for an outer canthus among the feature points extracted from the eye area image 403-1. The eye coordinate learner 120′ may use a line segment connecting the feature point 403-10 for the medial canthus and the feature point 403-11 for the outer canthus as a horizontal axis for the coordinates of the pupil center. The x-coordinate of the feature point 403-10 for the medial canthus and the x-coordinate of the feature point 403-11 for the outer canthus may correspond to both extreme values of the horizontal axis. The difference between the x-coordinate of the feature point 403-10 for the medial canthus and the x-coordinate of the feature point 403-11 for the outer canthus may refer to the entire length of the horizontal axis. The eye coordinate learner 120′ may calculate the horizontal coordinates of the normalized pupil center using the horizontal coordinate values of the pupil center compared to the length of the entire horizontal axis.

In addition, the eye coordinate learner 120′ may extract vertical coordinates of the normalized pupil center using feature points related to upper and lower eyelids among the feature points extracted from the eye area image 403-1. In this case, as the feature point related to the upper eyelid, a feature point 403-12 with the largest y-axis coordinate among the feature points extracted from the eye may be used. Hereinafter, the feature point 403-12 will be referred to as an upper eyelid feature point.

As the feature point related to the lower eyelid, a feature point 403-13 with the smallest y-axis coordinate among the feature points extracted from the eye may be used. Hereinafter, the feature point 403-13 will be referred to as a lower eyelid feature point.

The eye coordinate learner 120′ may use a line segment connecting the upper eyelid feature point 403-12 and the lower eyelid feature point 403-13 as a vertical axis for the coordinates of the pupil center. The y-coordinate of the upper eyelid feature point 403-12 and the y-coordinate of the lower eyelid feature point 403-13 may correspond to the two extreme values of the vertical axis. The difference between the y-coordinate of the upper eyelid feature point 403-12 and the y-coordinate of the lower eyelid feature point 403-13 may refer to the length of the entire vertical axis. The eye coordinate learner 120′ may calculate the horizontal coordinates of the normalized pupil center using the vertical coordinate values of the pupil center compared to the length of the entire vertical axis. This corresponds to an example and the present disclosure is not limited to the feature point.

In FIG. 17, the feature point 403-10 for the medial canthus and the feature point 403-11 for the outer canthus are illustrated as being positioned on the same horizontal line, and the upper eyelid feature point 403-12 and the lower eyelid feature point 403-13 are illustrated as being positioned on the same vertical line. However, this may vary depending on a capturing angle of a camera, a head angle of a persona, etc.

For example, in the case of the eye area image rotating 30° clockwise based on the eye area image 403-10, the feature point 403-10 for the medial canthus and the feature point 403-11 for the outer canthus may not be positioned on the same horizontal line, and the upper eyelid feature point 403-12 and the lower eyelid feature point 403-13 may not be positioned on the same vertical line. In this case, the eye coordinate learner 120′ may transform the image so that in the rotating eye area image, the feature point 403-10 for the medial canthus and the feature point 403-11 for the outer canthus are positioned on the same horizontal line, and the upper eyelid feature point 403-12 and the lower eyelid feature point 403-13 are positioned on the same vertical line. In this case, the eye coordinate learner 120′ may transform the rotating eye area image using an affine transform, etc., and this is an example, and the present disclosure is not limited thereto.

The eye coordinate learner 120′ may extract the coordinates of the feature points and the eye feature points from each eye area image 403-1 or 403-2, and generate the coordinate information of the normalized pupil centers for the frame images of each video. The eye coordinate learner 120′ may train the second artificial neural network model further using the horizontal coordinate values and the vertical coordinate values of the normalized pupil center for the generated frame images of each video as the training data.

In addition, the eye coordinate learner 120′ may calculate the average value of the horizontal coordinate value and the vertical coordinate value of the pupil center calculated from the multi-frame image. The eye coordinate learner 120′ may train the second artificial neural network model further using the average value of the horizontal coordinate values and the vertical coordinate values of the pupil center as training data. The eye coordinate learner 120′ may train the second artificial neural network model to generate the information related to the reference coordinates of a pupil center in which the difference in coordinates of the pupil center is corrected by the position and angle of the camera using the average value.

The information related to the coordinates of the pupil center may include the vertical coordinate values and the horizontal coordinate values of the pupil center, the movement information of the pupil center in the vertical direction and the movement information of the pupil center in the horizontal direction, etc., according to the frame image of the m-th video. The information related to the coordinates of the pupil center may include contents about the two-dimensional coordinates.

The eye coordinate learner 120′ may train the second artificial neural network model to generate the information related to the coordinates of the pupil center of the left eye and the information related to the coordinates of the pupil center of the right eye in the frame image of the m-th video by extracting the eye area images for the left eye and the right eye.

According to another exemplary embodiment of the present specification, the memory 100′ may store data of at least one virtual object. The second artificial neural network model may be trained to generate information related to the coordinates of the pupil center by allowing the eye coordinate learner 120′ to use training data that includes a parameter value obtained by changing at least one of parameters related to head rotation, eye rotation, and camera settings of a parameter of a virtual object and an image of the virtual object acquired according to the parameter value.

Referring back to FIG. 7, the eye coordinate learner 120′ may change at least one of the parameters related to the head rotation, the eye rotation, and the camera settings of the virtual object. As an example, the eye coordinate learner 120′ may set parameter values so that the virtual camera captures the virtual object from the front (upper drawing of FIG. 7). The eye coordinate learner 120′ may change parameter values so that the virtual camera captures the virtual object from the right (lower drawing of FIG. 7). In addition, the eye coordinate learner 120′ may change parameter values for a distance between the virtual camera and the virtual object.

The eye coordinate learner 120′ may set parameter values so that the head of the virtual object rotates in at least one direction of a roll, a pitch, and a yaw.

The eye coordinate learner 120′ may set parameter values so that the eye of the virtual object rotates in at least one direction of the roll, the pitch, and the yaw.

The eye coordinate learner 120′ may change at least one of the parameters and acquire the image of the virtual object. The eye coordinate learner 120′ may train the second artificial neural network model using the parameter values and the virtual object according to the parameter values. In this case, the second artificial neural network model may be trained to generate the information on the two-dimensional coordinates and/or three-dimensional coordinates of the pupil center.

The virtual object may mean a Gaussian avatar generated using a 3D Gaussian splatter. The eye coordinate learner 120′ may control a latent vector of the virtual object to change Euler coordinates of the head and pupil of the virtual object. The Gaussian avatar corresponds to an example, and the present disclosure is not limited thereto, and a virtual object generated using a technique widely known among those skilled in the art may be used.

In addition, the memory 100′ may store labeling data including at least one of head coordinates, coordinates of a pupil center, and information related to camera settings according to an image in which a human face is captured. The eye coordinate learner 120′ may train the second artificial neural network model using the data.

In addition, the eye coordinate learner 120′ may train the second artificial neural network model using at least one of training data using the eye area image, training data obtained according to parameter changes of the virtual object, and labeling data.

According to an exemplary embodiment of the present specification, the third artificial neural network model may extract an eye area image, which is an image of an area including an eye extracted from each multi-frame image of multiple videos in which a human face is captured, by the eye rotation learner 130′. The eye rotation learner 130′ may train the third artificial neural network model to generate an eye rotation value using training data including information related to the eye phase changes generated according to the time sequence of the eye area image according to each video.

The eye rotation learner 130′ may generate the multi-frame image by controlling the sync of the multiple videos. Alternatively, the eye rotation learner 130′ may receive the multi-frame image generated from the head coordinate learner 110′.

The eye rotation learner 130′ may extract an eye area image for a frame image of the m-th video from the multi-frame image. The eye rotation learner 130′ may generate the information related to the eye phase changes by comparing the eye area image extracted from the multi-frame image with eye area images extracted from multi-frame images within a preset time range based on the multi-frame image. For example, the eye rotation learner 130′ may generate the information related to the eye phase changes by comparing the eye area image extracted from an arbitrary multi-frame image with the eye area images extracted from the multi-frame image acquired within 0.1 seconds or so based on the multi-frame image. In this case, the eye rotation learner 130′ may generate the information related to the eye phase changes according to the m-th video using the eye area image for the m-th video. This corresponds to an example and the present disclosure is not limited to the time.

According to an exemplary embodiment of the present specification, the eye rotation learner 130′ may extract an iris area image, which is an image of an area occupied by an iris, from the eye area image. The iris area image may refer to the iris area image in the frame image of the m-th video. The iris area image may refer to an image inside a bounding box including an iris in the eye area image. The eye rotation learner 130′ may train the third artificial neural network model using information related to a phase change of the iris according to the time sequence of the iris area image.

More specifically, the eye rotation learner 130′ may compare an iris area image extracted from an arbitrary multi-frame image with iris area images extracted from multi-frame images within a preset time range based on an arbitrary multi-frame image. The iris area image may be a mask image in which an area occupied by an iris is distinguished by different pixel values from other areas, which is an example, and the present disclosure is not limited thereto.

In addition, the eye rotation learner 130′ may generate the information related to the phase change using the mask image of the iris generated by the eye coordinate learner 120′.

The eye rotation learner 130′ may generate the information related to the phase change of the iris by comparing the pixel values of the iris area image extracted from the arbitrary multi-frame image with those of other iris area images. The eye rotation learner 130′ may generate phase cross correlation values for the pixel values of the iris area image extracted from the arbitrary multi-frame image and other iris area images by using phase cross correlation analysis. The information related to the phase change calculated by using the phase cross correlation analysis may include contents about the change in the angle of the iris.

The eye rotation learner 130′ may generate the phase cross correlation value by a method of obtaining a cross correlation value upsampled by a fast Fourier transform (FFT). The eye rotation learner 130′ may calculate an initial estimate value of a cross correlation peak using the FFT, and then generate the phase cross correlation value by precisely estimating a phase shift of the upsampled signal using the discrete Fourier transform (DFT) in a preset area based on the estimated value. This corresponds to an example and the present disclosure is not limited to the method.

The eye rotation learner 130′ may train the third artificial neural network model using the image and the phase cross correlation value of the iris area as the training data.

According to an exemplary embodiment of the present specification, the eye rotation learner 130′ may calculate the size of the area occupied by the pupil in the eye area image. The eye rotation learner 130′ may adjust the size of the target eye area image according to the preset criteria. The target eye area image refers to an eye area image extracted from the arbitrary multi-frame image, and is not a term referring to a specific eye area image.

The eye rotation learner 130′ may compare the sizes of the areas occupied by the pupils extracted from the target eye area image extracted from the arbitrary multi-frame image and the preceding eye area image extracted from the immediately preceding multi-frame image. The eye rotation learner 130′ may adjust the size of the target eye area image so that the size of the area occupied by the pupil extracted from the target eye area image has a value within a preset difference value from the size of the area occupied by the pupil extracted from the preceding target eye area image. In this case, the eye rotation learner 130′ may adjust the size of the target eye area image extracted from the m-th video by comparing the sizes of the areas occupied by the pupils extracted from the eye area image corresponding to the m-th video. The eye rotation learner 130′ may calculate the phase cross correlation value after adjusting the sizes of each eye area image.

In addition, the eye rotation learner 130′ may generate a bounding box of an area including an eye in the multi-frame image. The eye rotation learner 130′ may extract the pupil center within the bounding box. The eye rotation learner 130′ may adjust the bounding box so that the pupil center is positioned at the center of the bounding box. Alternatively, the eye rotation learner 130′ may receive the information related to the coordinates of the pupil center generated from the second artificial neural network model.

The eye rotation learner 130′ may extract an image inside the adjusted bounding box as the eye area image. The eye rotation learner 130′ may extract the iris area image after adjusting the size of the eye area image according to the method described above and may calculate the phase cross correlation value.

The eye rotation learner 130′ may train the third artificial neural network model to generate the eye rotation value for the m-th video using the iris area image and the phase cross correlation value. The eye rotation value may refer to an angle of rotation clockwise or counterclockwise based on the central axis of the eye. In addition, the eye rotation learner 130′ may calculate an average value of eye rotation values calculated from the frame images in the m-th video over time. The eye rotation learner 130′ may train the third artificial neural network model to generate the reference eye rotation value using the average value. The reference eye rotation value may refer to a rotation value in which the difference in the rotation value calculated from each video is corrected according to the position, angle, etc., of the camera.

The eye rotation learner 130′ may train the third artificial neural network model to generate the information related to the phase changes of the left and right eyes by extracting the eye area images for the left and right eyes from the frame image.

According to an exemplary embodiment of the present specification, the eye coordinate learner 120′ may train the second artificial neural network model using the information generated from the first artificial neural network model. In addition, the eye rotation learner 130′ may train the third artificial neural network model using the information generated from the second artificial neural network model.

For example, the eye coordinate learner 120′ may generate the training data using the information related to the head coordinates in the frame images of the m-th video extracted from the plurality of multi-frame images input to the first artificial neural network model and each multi-frame image. The eye coordinate learner 120′ may generate the eye area image from each multi-frame image using the information related to the head coordinates extracted from each multi-frame image. The eye coordinate learner 120′ may generate the eye area images from each multi-frame image using the information related to the eye coordinates from the information related to the head coordinates. The eye coordinate learner 120′ may train the second artificial neural network model according to the process described above.

The eye rotation learner 130′ may train the third artificial neural network model further using the information related to the coordinates of the pupil center according to each multi-frame image generated from the second artificial neural network model. In addition, the eye rotation learner 130′ may train the third artificial neural network model by generating the training data according to the process described above using the image of the iris and/or pupil segmented by the eye coordinate learner 120′.

The first to third artificial neural network models may be trained independently from each other, and may also be trained using the information generated from each artificial neural network model.

Hereinafter, the process in which the balance function management system 10′ generates the balance function status information and performs the balance function rehabilitation program using the trained artificial neural network model will be described.

The balance function management system 10′ may acquire frame images of n videos in real time from n cameras that capture a subject performing a balance function status check and/or a balance function rehabilitation program.

The balance function management system 10′ may control the sync of the plurality of cameras through at least one processor. For example, the at least one processor may control the sync of the plurality of cameras in real time using a technology such as Genlock, which is an example, and the present disclosure may control the sync of the plurality of cameras through a technology widely known to those skilled in the art.

In addition, the balance function management system 10′ may sample frame images of the plurality of cameras through at least one processor. For example, one camera may capture the subject at 100 FPS, and another camera may capture the subject at 50 FPS. In this case, the balance function management system 10′ may down-sample a video captured at 100 FPS by ½ times or up-sample a video captured at 50 FPS by 2 times using at least one processor. This is an example, and the present disclosure is not limited thereto.

Preferably, the balance function management system 10′ may acquire a video of capturing a subject performing the balance function status check and/or the balance function rehabilitation program in real time through the plurality of cameras having the same FPS setting value.

The balance function management system 10′ may acquire at least one of the head coordinates, the coordinates of the pupil center, and the information related to the eye phase changes of the subject using at least one of the first to third artificial neural network models.

The balance function management system 10′ may acquire the head coordinates, the coordinates of the pupil center, and/or the information related to the eye phase changes using the first to third artificial neural network models.

In addition, the balance function management system 10′ may generate the information related to the head coordinates, the coordinates of the pupil center, and the eye phase changes using an algorithm in which at least one processor generates the training data of the first to third artificial neural network models described above.

Hereinafter, it will be described that the information related to the head coordinates, the coordinates of the pupil center, and the eye phase changes is generated by using the first to third artificial neural network models. However, it is not necessary to use the artificial neural network model to generate the information.

The head coordinate acquirer 140′ may execute the first artificial neural network model stored in the memory 100′ and input the multi-frame image concatenating the frame images of n videos to the first artificial neural network model to acquire the information related to the head coordinates according to the m-th video. The information related to the head coordinates according to the m-th video may refer to the information related to the head coordinates for the m-th video generated according to the order of the multi-frame images.

The eye coordinate acquirer 150′ may execute the second artificial neural network model stored in the memory 100′ and input the information related to the head coordinates to the second artificial neural network model to acquire the information related to the coordinates of the pupil center according to the m-th video. The information related to the coordinates of the pupil center according to the m-th video may refer to the information related to the coordinates of the pupil center of the m-th video generated according to the order of the multi-frame images.

The phase change acquirer 160′ may execute the third artificial neural network model stored in the memory 100′ and input the information related to the coordinates of the pupil center of the m-th video according to the time sequence of the multi-frame images to the third artificial neural network model to acquire the information related to the eye phase changes according to the m-th video. The information related to the eye phase changes according to the m-th video may refer to the information related to eye phase changes generated according to the order of the frame images of the m-th video.

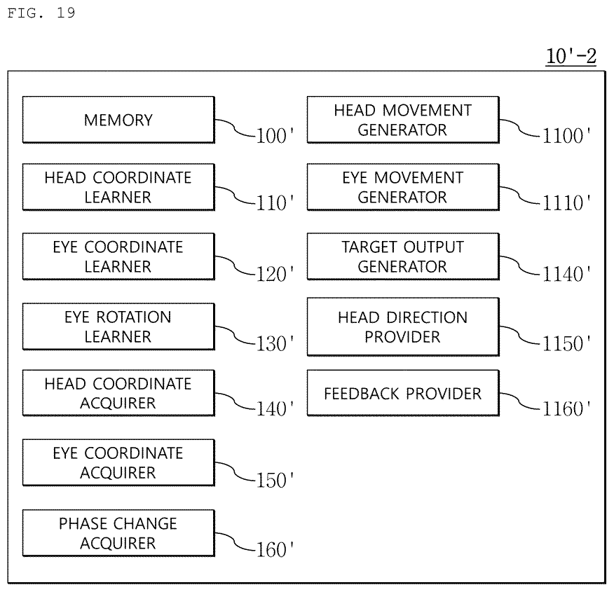

FIG. 18 is a block diagram of a balance function management system for generating information on a balance function status according to an exemplary embodiment of the present specification.

Referring to FIG. 18, a balance function management system 10′-1 for generating balance function status information according to an exemplary embodiment of the present specification may include the memory 100′, the head coordinate learner 110′, the eye coordinate learner 120′, the eye rotation learner 130′, the head coordinate acquirer 140′, the eye coordinate acquirer 150′, the phase change acquirer 160′, a head movement generator 1100′, an eye movement generator 1110′, a speed information generator 1120′, and a balance function status information generator 1130′.

Since the memory 100′, the head coordinate learner 110′, the eye coordinate learner 120′, the eye rotation learner 130′, the head coordinate acquirer 140′, the eye coordinate acquirer 150′, and the phase change acquirer 160′ have been described above, a repetitive description thereof will be omitted.

The head movement generator 1100′ may generate the information related to the head movement in the m-th video using the information related to the head coordinates acquired from the first artificial neural network model. The information related to the head movement may include horizontal movement and vertical movement of a head, and a degree of rotation of a head over time. The degree of rotation of the head may refer to a rotation angle of a head in the roll, pitch, and yaw directions. The information related to the head movement may be expressed as a graph of horizontal coordinate values and vertical coordinate values of the head over time.

According to an exemplary embodiment of the present specification, the head movement generator 1100′ may calculate a normal vector of a subject's head using the feature points of the head generated in each frame image and the coordinates of the feature points. The direction of the normal vector may refer to the direction in which the front of the subject's head faces. The direction in which the front of the subject's head faces may refer to the direction in which the tip of the nose faces. The head movement generator 1100′ may calculate a normal vector of the head using the feature points of the head, based on the feature point of the tip of the nose among the feature points of the head.

Alternatively, the direction in which the front of the subject's head faces may refer to the direction in which any feature point (such as the tip of the forehead or the center of the lips) that is on a straight line vertically based on the feature point of the tip of the nose faces. The head movement generator 1100′ may calculate the normal vector of the head based on any one of the feature points.

Since calculating the normal vector for the front of the head using the feature point is a widely known technique among those skilled in the art, a detailed description thereof will be omitted.

The head movement generator 1100′ may generate the information related to the head movement over time using the 3D head coordinate information and the normal vector according to the frame image of the m-th video. The head movement generator 1100′ may output the information related to the head movement as a graph.

The eye movement generator 1110′ may generate the information related to the eye movement in the m-th video using the information related to the coordinates of the pupil center and the eye phase changes generated from the second artificial neural network model and the third artificial neural network model. The information related to the eye movement may include the information related to the movement of the pupil center in the vertical direction, the movement of the pupil center in the horizontal movement, and the rotation value of the eye over time. The information related to the eye movement may be expressed as a graph of vertical coordinate value, horizontal coordinate value, and rotation angle of the pupil center of left and right eyes over time.

The eye movement generator 1110′ may calculate a gaze vector of an eye using the vertical coordinate value, horizontal coordinate value, and rotation value of the pupil center. Since calculating the gaze vector using the coordinate value and rotation value of the pupil center is a widely known technique among those skilled in the art, a detailed description thereof will be omitted.

The eye movement generator 1110′ may output the information related to the eye movement as a graph.

According to an exemplary embodiment of the present specification, the head movement generator 1100′ and the eye movement generator 1110′ may correct errors between the head movement and the eye movement information according to the training data and the head movement and the eye movement information of the subject.

The head movement and the eye movement information according to the training data may refer to actual data values for training the artificial neural network model.

For example, the actual data values may refer to parameter values of the Euler angles of the head and eyes acquired by the eye coordinate learner 120′. The eye coordinate learner 120′ may set parameter values of the virtual camera to be similar to actual settings in the balance function status check and/or the balance function rehabilitation program. The eye coordinate learner 120′ may change parameter values of the Euler angle to be similar to head and eye movements of the subject according to the balance function status check and/or the balance function rehabilitation program. In this case, the head and eye movement information of the virtual object according to the change in the parameter values of the head and eyes may refer to actual data values. This is an example, and the actual data value may refer to the head and eye movement information according to the actual data value that may be used to train the first to third artificial neural network models.

The head movement generator 1100′ and the eye movement generator 1110′ may correct the errors in the head movement and the eye movement between the frame images acquired within a preset time. For example, when the preset time is 1.5 seconds and the camera captures the subject at 100 FPS, the head movement generator 1100′ and the eye movement generator 1110′ may correct the errors in the head movement and the eye movement between 150 frame images. This is an example, and the present disclosure is not limited to the time and frame rate.

The head movement generator 1100′ and the eye movement generator 1110′ may correct the errors of the head movement and the eye movement to have values within a preset range.

For example, the head movement generator 1100′ and the eye movement generator 1110′ may generate the information related to the head movement and the eye movement using the information related to the head coordinates, the coordinates of the pupil center, and/or the eye phase changes acquired from 150 frame images (frame images acquired for 1.5 seconds based on an arbitrary frame image) in the video in which the plurality of cameras captures the subject at 100 FPS. The information related to the head movement and the eye movement may be calculated as the amount of head and eye movement (vertical, horizontal, rotation) over time according to the order of the frame images. In this case, the amount of head and eye movement at the time corresponding to the 50th frame image (frame image acquired 0.5 seconds after an arbitrary frame image) may be outside the preset error range. In this case, the head movement generator 1100′ and the eye movement generator 1110′ may calculate statistical values, such as the average or median of the amount of head and eye movement generated using information from the preceding frame image to replace the amount of movement at the time corresponding to the 50th frame image.

Although it is described that the error is corrected between frame images acquired for 1.5 seconds based on an arbitrary frame image, this is an example, and various exemplary embodiments, such as frame images acquired 1.5 seconds before the arbitrary frame image and frame images acquired within 1.5 seconds or so, may occur, and this is an example, and the present disclosure is not limited to the time, frame rate, statistical values, etc.

As another example, the head movement generator 1100′ and the eye movement generator 1110′ may correct the errors of the head and eye movements by applying a filter. For example, the head movement generator 1100′ and the eye movement generator 1110′ may correct the errors using filters such as a chaining Kalman filter, a moving average filter, a Savitzky-Golay filter, a high pass filter, a low pass filter, and a band pass filter, which is an example, and the present disclosure is not limited thereto, and various types of filters may be used.

According to another exemplary embodiment of the present specification, the head coordinate learner 110′, the eye coordinate learner 120′, and the eye rotation learner 130′ may train the first to third artificial neural network models to correct the error. The first to third artificial neural networks may generate the information related to the head coordinates, the coordinates of a pupil center, and the eye phase changes for which the error is corrected.

The speed information generator 1120′ may generate the information related to the head and eye movement speeds in the m-th video using the information related to the head movement and the eye movement. The speed information generator 1120′ may calculate the vertical movement speed of the head, the horizontal movement speed of the head, and/or the rotation speed of the head over time using the information related to the head movement. The speed information generator 1120′ may calculate the vertical movement speed of the eye, the horizontal movement speed of the eye, and/or the rotation speed of the eye over time using the information related to the eye movement.

According to an exemplary embodiment of the present specification, the speed information generator 1120′ may filter out noise values from the information related to the head and eye movement speeds. For example, when performing the balance function status check, noise values may occur in which the speeds of the head and eye movements are not accurately calculated by the case where the subject's eyes are covered, the case where the subject rotates his/her head quickly or slowly, or the case where the head position changes, etc. The speed information generator 1120′ may remove noise from the information related to the head and eye movement speeds using the filters such as a chaining Kalman filter, a moving average filter, a Savitzky-Golay filter, a high pass filter, a low pass filter, or a band pass filter, and this is only an example, and the present disclosure is not limited thereto, and various noise processing methods may be used.

According to an exemplary embodiment of the present specification, the speed information generator 1120′ may generate the information related to the head movement speed and the eye movement speed within a preset time based on a time when the head movement becomes greater than or equal to a preset threshold value. In the balance function status check, the subject may move the head in a horizontal (lateral left, lateral right) direction. In this case, the speed information generator 1120′ may generate the information related to the head and eye movement speeds when the movement of the head in the horizontal direction is greater than or equal to the preset threshold value.

In addition, the subject may move the head in the lower right and upper right directions while the right side of the face is turned toward the front of the subject so that a right anterior semicircular canal and a left posterior semicircular canal (right anterior, left posterior (RALP)) are stimulated. In addition, the subject may move the head in the left-down and left-up directions while the left side of the face is turned toward the front of the subject so that the right posterior semicircular canal and the left anterior semicircular canal (left anterior, right posterior (RALP)) are stimulated. In this case, the speed information generator 1120′ may generate the information related to the head and eye movement speeds when the movement of the head in the vertical direction is greater than or equal to the preset threshold value.

Hereinafter, the direction in which the subject rotates the head so that the RALP is stimulated will be described as the RALP direction, and the direction in which the subject rotates the head so that the LARP is stimulated will be described as the LARP direction.

For example, the speed information generator 1120′ may determine whether the head movement is greater than or equal to a threshold value by using the head movement information according to a frame image existing within a preset time based on the last input frame image. The speed information generator 1120′ may calculate the difference between the maximum and minimum values of the coordinates of the feature points of the head in the head movement information according to the frame image existing within the preset time to determine whether the head movement is greater than or equal to the threshold value. The preset threshold value may vary depending on the frame rate of the video, the size of the frame image, etc.

As another example, the memory 100′ may further store a fourth artificial neural network model that generates the information on the head movement. The fourth artificial neural network model may be trained by allowing at least one processor to use the frame image of the video performing the balance function status check and the data of the pitch and yaw values of the head in the corresponding frame image. The fourth artificial neural network model may be a time series model or a transformer model, which is an example, and the present disclosure is not limited to the model. In this case, the frame image may be labeled with information on the lateral, RALP, and LARP directions. The speed information generator 1120′ may input a frame image existing within a preset time based on the last acquired frame image to the fourth artificial neural network model to confirm whether the head movement is greater than or equal to the threshold value.

The speed information generator 1120′ may calculate the head and eye movement speeds using the information related to the head and eye movement generated during a preset time after the time when the head movement becomes greater than or equal to the threshold value. For example, the speed information generator 1120′ may calculate the head and eye movement speeds using the information related to the head and eye movement generated within 1.5 seconds after the time when the head movement becomes greater than or equal to the threshold value, which is an example, and the present disclosure is not limited to the time. The speed information generator 1120′ may calculate the head and eye movement speeds in the m-th video, respectively.

The speed information generator 1120′ may display the information related to the head and eye movement speeds on the display device. The speed information generator 1120′ may display the information related to the head and eye movement speeds from which noise has been removed and/or the information related to the head and eye movement speeds from which noise has not been removed on the display.

The balance function status information generator 1130′ may generate the information on the balance function status of the subject using the information related to the head and eye movement speeds.

According to an exemplary embodiment of the present specification, the balance function status information generator 1130′ may calculate a gain coefficient using a time value (head peak index) when the head movement speed of the subject is relatively the largest within a preset analysis window and a time value (eye peak index) when the eye movement speed is relatively the largest when the eye of the subject moves in the direction of the head movement and then returns to the original position. The analysis window may mean a preset time range based on the time when the head movement becomes greater than or equal to the threshold value. The size of the analysis window may correspond to a time range in which the speed information generator 1120′ generates the information related to the head and eye movement speeds.

For example, the size of the analysis window may be 1.5 seconds after the time when the head movement becomes greater than or equal to the threshold value, which is an example, and the present disclosure is not limited thereto.

The balance function status information generator 1130′ may calculate the gain coefficient using the above [Equation 1] using the head peak index and the eye peak index from the information related to the head and eye movement speeds from which noise has been removed.

Thereafter, the balance function status information generator 1130′ may calculate gain using the above [Equation 2].

The balance function status information generator 1130′ may calculate the gains for the left eye and the right eye, respectively.

The balance function status information generator 1130′ may calculate the gains in the m-th video, respectively. For example, when the subject is captured using two cameras, the gains for the two videos may be calculated, respectively. In this case, the gains of the left eye and the right eye in the first video and the gains of the left eye and the right eye in the second video may be calculated. The balance function status information generator 1130′ may calculate a statistical value for the gain of the left eye calculated in the first video and the second video, and calculate at least one statistical value for the gain of the right eye calculated in the first video and the second video. The statistical value may correspond to an average, a median, a minimum, a maximum, a standard deviation, etc., and this is an example, and the present disclosure is not limited thereto.

According to an exemplary embodiment of the present specification, the head movement generator 1100′ may further generate reference head movement information by calculating statistical values of the information related to the head coordinates according to the m-th video. The head movement generator 1100′ may further generate the reference head movement information by calculating the statistical values of the information related to the head coordinates according to the m-th video generated from the multi-frame image.

The eye movement generator 1110′ may further generate the reference eye movement information by calculating the statistical values of the information related to the coordinates of a pupil center and the eye phase changes according to the m-th video. The eye movement generator 1110′ may further generate the reference eye movement information by calculating the statistical values of the information related to the coordinates of a pupil center and the eye phase changes according to each video generated from the multi-frame image.

When the subject is captured by the plurality of cameras, a difference in the coordinate values of the 3D head generated from the frame images of the m-th video may occur depending on the position, angle, etc., of the cameras.

For example, a first camera may capture the subject from the right side of the subject, and a second camera may capture the subject from the left side of the subject. In this case, when the subject turns his/her head to the right, the coordinates of the feature points positioned on the right side of the face of the subject may be calculated more accurately than the coordinates of the feature points positioned on the left side in the frame image of the first video captured by the first camera. In addition, when the subject turns his/her head to the left, the coordinates of the feature points positioned on the left side of the face of the subject may be calculated relatively more accurately than the coordinates of the feature points positioned on the right side in the frame image of the second video captured by the second camera.

As another example, a plurality of cameras may be installed to surround the subject at 15° intervals at a distance of 1 m from the subject. In this case, the plurality of cameras may capture the subject at an eye height of the subject. Even in this case, the coordinates of the feature points that are measured relatively more accurately for each camera may be generated depending on the direction of the head movement of the subject.

In this way, the information of the two-dimensional head coordinates generated depending on the positions, angles, etc., of the plurality of cameras may be different from each other.

The head movement generator 1100′ may generate the reference head coordinate information by calculating the average value of the three-dimensional head coordinates generated from the frame images that are synchronized with each other in the m-th video. The head movement generator 1100′ may further generate the information related to the reference head movement using the reference head coordinate information over time.

Alternatively, the head coordinate acquirer 110′ may acquire the reference head coordinates from the first artificial neural network model. The head movement generator 1100′ may further generate the information related to the reference head movement using the reference head coordinates.

The eye movement generator 1110′ may generate the information related to the reference coordinates of the pupil center and the reference eye phase change by calculating an average value of the information related to the coordinates of the pupil center and the eye phase change generated from the frame images that are synchronized in the m-th video. The eye movement generator 1110′ may generate a reference gaze vector of the eye using the information related to the reference coordinates of the pupil center and the reference eye phase change. The eye movement generator 1110′ may further generate the information related to the reference eye movement using the reference coordinates of the pupil center, the information related to the reference eye phase change, and the reference gaze vector.

Alternatively, the eye coordinate acquirer 150′ and the phase change acquirer 160′ may acquire the information related to the reference coordinates of a pupil center and the rotation value information of the reference eye from the second and third artificial neural network models. The eye movement generator 1110′ may generate the information related to the reference eye movement using the information related to the reference coordinates of a pupil center and the rotation value information of the reference eye.

In this case, the speed information generator 1120′ may generate the information related to the head movement speed and the eye movement speed for the m-th video, respectively, within a preset time based on the time when the reference head movement becomes greater than or equal to the preset threshold value.

The balance function status information generator 1130′ may calculate the gains, respectively, using the head peak index and the eye peak index for the m-th video within the preset time based on the time when the reference head movement becomes greater than or equal to the preset threshold value.

As illustrated in FIG. 9, the head movement generator 1100′, the eye movement generator 1110′, the speed information generator 1120′, and the balance function status information generator 1130′ may output the calculated information on the display screen. The display screen may output videos captured by the plurality of cameras, respectively.

The speed information generator 1120′ may output the information on the head and eye movement speeds for the m-th video as a graph 205, respectively. In the graph 205 of the head and eye movement speeds, the speed information according to the number of times of balance function status checks may be superimposed and displayed. In the speed graph 205 of the head and eye movements, the time values at which a peak and/or valley appear may correspond to the head peak index and/or the eye peak index. In the graph, the vertical axis may correspond to the speed value, and the horizontal axis may correspond to the time value. Although the graph for the horizontal speed of the head and eye is illustrated in FIG. 9, this is an example, and graphs for the speed in the vertical and rotation directions may be further outputted depending on the type of test.

The head movement generator 1100′ and the eye movement generator 1110′ may output the head and eye movement information as graphs. The head movement generator 1100′ and the eye movement generator 1110′ may output a movement graph 206 of the head and eye in a horizontal direction and a movement graph 207 of the head and eye in a vertical direction. In addition, the movement graph for the rotation direction of the head and/or eye may be further outputted. In addition, the head and eye movement graphs may include the movement information for the m-th video, and may include the reference head movement and reference eye movement information.

The balance function status information generator 1130′ may output information 208 related to the balance function status check for the subject to a display device. The information related to the balance function status check may include at least one of the rotation direction (lateral, RALP, LARP) of the head, a gain and standard deviation according to the head rotation direction, the number of balance function status checks according to the rotation direction of the head, the number of times of successful calculations of the gain, and the number of times of failures in the calculation of the gain.

The case where the calculation of the gain fails may occur when the subject moves the head faster than the standard of the test. In this case, the difference between the head peak index and the eye peak index may exceed the middle of the analysis window size. In this case, the balance function status information generator 1130′ may fail to calculate the gain. The balance function status information generator 1130′ may generate the information on the number of times of successful and failed calculations of the gain, thereby providing an effect in which the subject and/or the examiner may make a more accurate determination.

In addition, the balance function status information generator 1130′ may generate information on whether the semicircular canal is abnormal according to the gain. For example, the balance function status information generator 1130′ may generate information on the abnormality of the semicircular canal when the gains of the left and right eyes are lower than or equal to the preset value in the balance function status check.

In addition, the subject may rotate his/her head in the lateral direction in the balance function status check. In this case, when the difference in the gain of the left and right eyes when the subject turns his/her head to the left and to the right is greater than or equal to the preset value, the balance function status information generator 1130′ may generate abnormal information of the semicircular canal.

In addition, in the balance function status check, the subject may rotate his/her head in the RALP or LARP direction. In this case, when the difference in the gain of the left and right eyes when the subject rotates his/her head upward and downward is greater than or equal to the preset value, the balance function status information generator 1130′ may generate abnormal information of the semicircular canal.

In addition, the balance function status information generator 1130′ may further generate the information on whether there is an abnormality in the central balance nerve function and an abnormality in the peripheral balance nerve function by using the information related to the eye movement and the gain information.

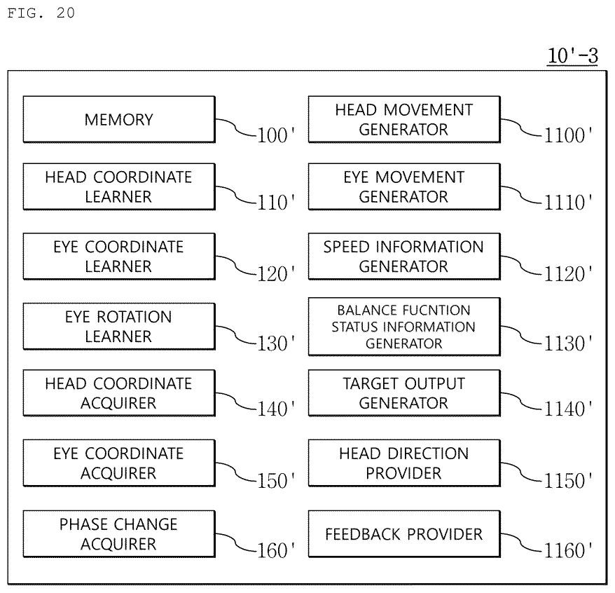

FIG. 19 is a block diagram of a balance function management system for performing a balance function rehabilitation program according to an exemplary embodiment of the present specification.

Referring to FIG. 19, a balance function management system 10′-2 for performing a balance function rehabilitation program according to an exemplary embodiment of the present specification may include the memory 100′, the head coordinate learner 110′, the eye coordinate learner 120′, the eye rotation learner 130′, the head coordinate acquirer 140′, the eye coordinate acquirer 150′, the phase change acquirer 160′, the head movement generator 1100′, the eye movement generator 1110′, a target output generator 1140′, a head direction provider 1150′, and a feedback provider 1160′. Since the memory 100′, the head coordinate learner 110′, the eye coordinate learner 120′, the eye rotation learner 130′, the head coordinate acquirer 140′, the eye coordinate acquirer 150′, the phase change acquirer 160′, the head movement generator 1100′, and the eye movement generator 1110′ have been described above, a repetitive description thereof will be omitted.

As illustrated in FIG. 11, the target output generator 1140′ may output a virtual target 209 to the display device. The virtual target may be displayed at any position on the display device. In FIG. 11, the target is illustrated in the shape of a trump card, but this is only an example, and the present disclosure is not limited to the shape.

The head direction provider 1150′ may provide the subject with the information on the rotation direction of the head according to the balance rehabilitation protocol. For example, the head direction provider 1150′ may provide information so that the subject rotates the head in the lateral direction.

Alternatively, the head direction provider 1150′ may provide information so that the subject rotates the head in the RALP or LARP direction.

In this case, the head direction provider 1150′ may provide information so that the subject rotates the head only upward or downward while the right side of the head is facing forward. In addition, the head direction provider 1150′ may provide information so that the subject rotates the head only upward or downward while the left side of the head is facing forward.

In addition, the head direction provider 1150′ may provide information so that the subject returns to a state before rotating the head within a preset time after rotating the head. For example, the head direction provider 1150′ may provide information to return to a state before rotating the head after rotating the head within 1 second.

The head direction provider 1150′ may visually display the information on the display device. In addition, the head direction provider 1150′ may output the information to an audio device.

The feedback provider 1160′ may provide feedback according to the head movement and the eye movement of the subject.

According to the balance rehabilitation protocol, the reference of the angle at which the subject should rotate the head may be determined in advance. The feedback provider 1160′ may compare the rotation angle of the subject's head generated from the head movement generator 1100′ with the reference of the angle. The feedback provider 1160′ may provide auditory feedback and/or visual feedback when the rotation angle of the subject's head satisfies the reference of the angle. In addition, the feedback provider 1160′ may provide auditory feedback and/or visual feedback when the rotation angle of the subject's head does not satisfy the reference of the angle. The feedback provider 1160′ may provide different feedbacks depending on whether the rotation angle of the subject's head satisfies the reference of the angle.

In addition, the eye movement generator 1110′ may generate the coordinate information of the gaze point which the subject's gaze faces on the display device using the eye gaze vector. The feedback provider 1160′ may compare the coordinate information of the gaze point generated from the eye movement generator 1110′ with the coordinate information of the virtual target 209.

When the coordinates of the gaze point are positioned within the area of the virtual target 209, the feedback provider 1160′ may change the color value of the virtual target 209. In addition, the feedback provider 1160′ may further display the gaze point within the virtual target 209.

When the coordinates of the gaze point are positioned outside the area of the virtual target 209, the feedback provider 1160′ may display the position of the gaze point on the display device.

When the subject is captured by the plurality of cameras, the head movement generator 1100′ and the eye movement generator 1110′ may generate the information related to the reference head movement and the reference eye movement as described above. The feedback provider 1160′ may provide the feedback according to the information related to the reference head movement and the reference eye movement.

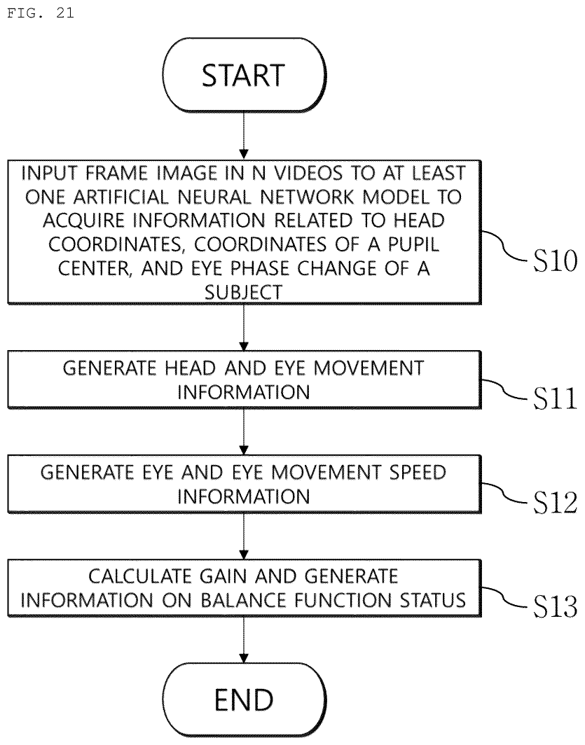

FIG. 20 is a block diagram of a balance function management system for generating information on a balance function status and performing a balance function rehabilitation program according to an exemplary embodiment of the present specification.

Referring to FIG. 20, a balance function management system 10′-3 may include the memory 100′, the head coordinate learner 110′, the eye coordinate learner 120′, the eye rotation learner 130′, the head coordinate acquirer 140′, the eye coordinate acquirer 150′, the phase change acquirer 160′, the head movement generator 1100′, the eye movement generator 1110′, the speed information generator 1120′, the balance function status information generator 1130′, the target output generator 1140′, the head direction provider 1150′, and the feedback provider 1160′. The balance function management system 10-3′ may generate a gain to provide a balance function rehabilitation program according to whether the balance function is abnormal.

The head coordinate learners 110 and 110′, the eye coordinate learners 120 and 120′, the eye rotation learners 130 and 130′, the head coordinate acquirers 140 and 140′, the eye coordinate acquirers 150 and 150′, the phase change acquirers 160 and 160′, the head movement generators 1100 and 1100′, the eye movement generators 1110 and 1110′, the speed information generators 1120 the balance function status and 1120′, information generators 1130 and 1130′, the target output generators 1140 and 1140′, the head direction providers 1150 and 1150′, and the feedback providers 1160 and 1160′ may include a processor, an application-specific integrated circuit (ASIC), other chipsets, logic circuits, registers, communication modems, data processing units, etc., that are known in the technical field to which the present disclosure pertains to perform calculation and various control logics. In addition, when the above-described control logic is implemented as software, the head coordinate learners 110 and 110′, the eye coordinate learners 120 and 120′, the eye rotation learners 130 and 130′, the head coordinate acquirers 140 and 140′, the eye coordinate acquirers 150 and 150′, the phase change acquirers 160 and 160′, the head movement generators 1100 and 1100′, the eye movement generators 1110 and 1110′, the speed information generators 1120 and 1120′, the balance function status information generators 1130 and 1130′, the target output generators 1140 and 1140′, the head direction providers 1150 and 1150′, and the feedback providers 1160 and 1160′ may be implemented as a set of program modules. In this case, the program module may be stored in the memory device and executed by the processor.

Hereinafter, a method of generating information on a balance function status and a balance function rehabilitation method using a balance function management system according to the present specification are disclosed. However, when describing the method of generating information on a balance function status and the balance function rehabilitation method according to the present specification, a repetitive description of each component will be omitted.

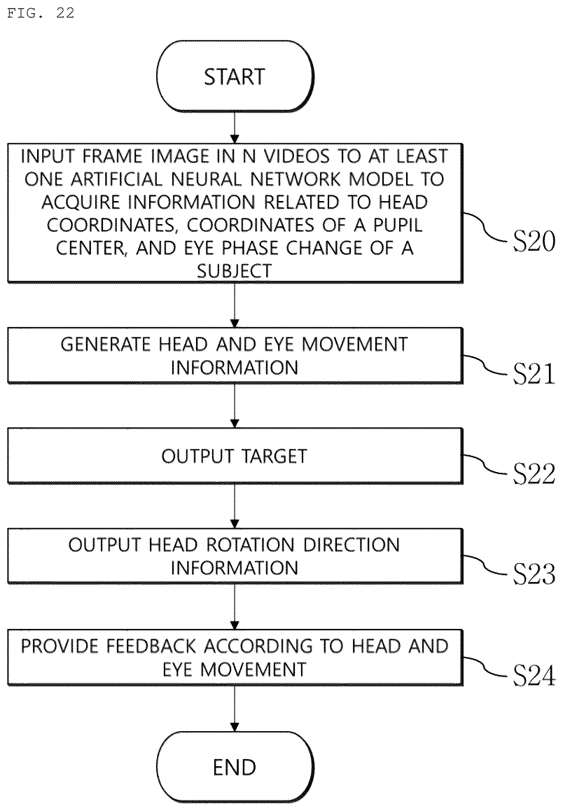

FIG. 21 is a flowchart of a method of generating information on a balance function status according to an exemplary embodiment of the present specification.

Referring to FIG. 21, in step S10, at least one processor may input frame images of n videos to the first artificial neural network model to acquire the information related to the head coordinates. The n videos may refer to videos of capturing a subject performing a balance function status check by n cameras. The frame images of the n videos may be sequentially input as described above, and the multi-frame images of the n videos may be input. Thereafter, the at least one processor may input the information related to the head coordinates of the n videos to the second artificial neural network model to acquire the information related to the coordinates of a pupil center. Thereafter, the at least one processor may input the information related to the coordinates of a pupil center of the n videos to the third artificial neural network model to acquire the information related to the eye phase changes. Since the learning process of the first to third artificial neural network models has been described above, a repetitive description thereof will be omitted.

In step S11, the at least one processor may generate the head and eye movement information using the information related to the head coordinates, the coordinates of a pupil center, and the eye phase changes. The at least one processor may generate the head and eye movements information for the m-th video, respectively. Alternatively, the at least one processor may further generate the reference head movement information and the reference eye movement information for the m-th video.

In step S12, when the head movement of the subject in the m-th video is greater than or equal to the preset threshold value, the at least one processor may generate the speed information of the head and eye movements for the m-th video. In addition, when the reference head movement of the subject is greater than or equal to the preset threshold value, the at least one processor may generate the speed information of the head and eye movements for the m-th video.

In step S13, the at least one processor may calculate the gain using the above [Equation 1] and [Equation 2]. The at least one processor may generate the balance function state information using the gain.

FIG. 22 is a flowchart of a balance function rehabilitation method according to an exemplary embodiment of the present specification.

Referring to FIG. 22, in step S20, at least one processor may input frame images of n videos to the first artificial neural network model to acquire the information related to the head coordinates. The n videos may refer to videos of capturing a subject performing a balance function rehabilitation program by n cameras. The frame images of the n videos may be sequentially input as described above, and the multi-frame images of the n videos may be input. Thereafter, the at least one processor may input the information related to the head coordinates of the n videos to the second artificial neural network model to acquire the information related to the coordinates of a pupil center. Thereafter, the at least one processor may input the information related to the coordinates of a pupil center of the n videos to the third artificial neural network model to acquire the information related to the eye phase changes. Since the learning process of the first to third artificial neural network models has been described above, a repetitive description thereof will be omitted.

In step S21, the at least one processor may generate the head and eye movement information using the information related to the head coordinates, the coordinates of a pupil center, and the eye phase changes. The at least one processor may generate the head and eye movements information for the m-th video, respectively. In addition, the at least one processor may further generate the reference head movement information and the reference eye movement information for the m-th video. The at least one processor may generate coordinate information of a gaze point according to a subject's gaze using the reference eye movement information.

In step S22, the at least one processor may output a virtual target to a display.

In step S23, the at least one processor may provide information on a rotation direction of a head to a subject. Since the information on the rotation direction of the head has been described above, a repetitive description thereof will be omitted.

In step S24, the at least one processor may provide auditory feedback and/or visual feedback to a subject using the head and eye movement information.

The method of generating information on a balance function status and the balance function rehabilitation method may be implemented in the form of a computer program that is written to perform each step on a computer and recorded on a computer-readable recording medium. In order for the computer to read the program and execute the methods implemented as the program, the above-described computer program may include a code coded in a computer language such as C/C++, C#, JAVA, or machine language that the processor (CPU) of the computer may read through a device interface of the computer. Such a code may include functional code related to a function or such defining functions necessary for executing the methods and include an execution procedure related control code necessary for the processor of the computer to execute the functions according to a predetermined procedure. In addition, the code may further include a memory reference related code for which position (address street number) in an internal or external memory of the computer the additional information or media necessary for the processor of the computer to execute the functions is to be referenced at. In addition, when the processor of the computer needs to communicate with any other computers, servers, or the like positioned remotely in order to execute the functions, the code may further include a communication-related code for how to communicate with any other computers, servers, or the like using the communication module of the computer, what information or media to transmit/receive during communication, and the like.

The storage medium is not a medium that stores data therein for a while, such as a register, a cache, a memory, or the like, but refers to a medium that semi-permanently stores the data therein and is readable by an apparatus. Specifically, examples of the storage medium include, but are not limited to, ROM, RAM, CD-ROM, a magnetic tape, a floppy disk, an optical data storage device, and the like. That is, the program may be stored in various recording media on various servers accessible by the computer or in various recording media on the computer of the user. In addition, media may be distributed in a computer system connected by a network, and a computer-readable code may be stored in a distributed manner.

Although exemplary embodiments of the present specification have been described with reference to the accompanying drawings, those skilled in the art to which the present specification belongs will appreciate that various specific forms may be made without departing from the spirit or essential feature of the present disclosure. Therefore, it is to be understood that the exemplary embodiments described hereinabove are illustrative rather than being restrictive in all aspects.

What is claimed is:

1. A balance function management system, comprising:
at least one processor; and
a memory that stores instructions executable by the processor and stores at least one artificial neural network model executed on a computing device,
wherein the at least one processor inputs frame images of n videos of a subject captured by n (natural number) cameras to at least one artificial neural network model to acquire at least one of information related to head coordinates, coordinates of a pupil center, and eye phase changes of the subject according to an order of frame images of an m-th (natural number from 1 to n) video, and
uses the information to generate information related to head movement and eye movement for generating information on a balance function status or performing a balance function rehabilitation program.

2. The balance function management system according to claim 1, wherein the at least one processor comprising:
a head coordinate acquirer that executes a first artificial neural network model stored in the memory, and inputs frame images of the m-th video or multi-frame images concatenating frame images of n videos to the first artificial neural network model to acquire the information related to the head coordinates according to the m-th video;
an eye coordinate acquirer that executes a second artificial neural network model stored in the memory, and inputs the information related to the head coordinates to the second artificial neural network model to acquire information related to coordinates of a pupil center according to the m-th video; and
a phase change acquirer that executes a third artificial neural network model stored in the memory, and inputs information related to coordinates of a pupil center according to a time sequence of the multi-frame image or the frame image of the m-th video to the third artificial neural network model to acquire information related to the eye phase changes according to the m-th video.

3. The balance function management system according to claim 2, wherein the first artificial neural network model is an artificial neural network model trained by allowing the at least one processor to use, as training data, facial feature points extracted from frame images of at least one video in which a human face is captured or multi-frame images in which frame images of multiple videos in which a human face is captured are concatenated and coordinates of the feature points.

4. The balance function management system according to claim 3, wherein the feature point is a feature point positioned within a preset area in the frame images or the multi-frame images.

5. The balance function management system according to claim 2, wherein the second artificial neural network model is an artificial neural network model trained to generate the information related to the coordinates of a pupil center by allowing the at least one processor to use, as training data, eye area images extracted to include an eye from frame images of at least one video in which a human face is captured or multi-frame images in which frame images of multiple videos in which a human face is captured are concatenated.

6. The balance function management system according to claim 5, wherein the at least one processor generates pupil area images in which an area occupied by the pupil and the remaining area have different pixel values in the eye area images, and trains the second artificial neural network model using the pupil area images or an array of pixel values of the pupil area images as the training data.

7. The balance function management system according to claim 5, wherein the at least one processor trains the second artificial neural network model to generate eye feature points and coordinate information of the feature points from the eye area images, and to generate horizontal coordinate values and vertical coordinate values of the pupil center using coordinates of a plurality of preset feature points.

8. The balance function management system according to claim 2, wherein the memory stores data of at least one virtual object, and
the second artificial neural network model is an artificial neural network model trained to generate the information related to the coordinates of a pupil center by allowing the at least one processor to use training data that includes a parameter value that changes at least one of parameters related to head rotation, eye rotation, and camera settings of the virtual object and an image of the virtual object acquired according to the parameter value.

9. The balance function management system according to claim 2, wherein the third artificial neural network model is an artificial neural network model trained to generate an eye rotation value by allowing the at least one processor to use, as training data, information related to eye phase changes generated according to a time sequence of eye area images extracted to include an eye from frame images of at least one video in which a human face is captured or multi-frame images in which frame images of multiple videos in which a human face is captured are concatenated.

10. The balance function management system according to claim 9, wherein the third artificial neural network model is an artificial neural network model trained by allowing the at least one processor to use information comparing pixel values of an area occupied by an iris between eye area images corresponding to each frame image of each video, or to each frame image of each video in the multi-frame images.

11. The balance function management system according to claim 9, wherein the at least one processor calculates a size of an area occupied by a pupil in the eye area images and adjusts a size of a target eye area image using the size of the area occupied by the pupil in a preceding eye area image.

12. The balance function management system according to claim 1, wherein the at least one processor comprising:
a head movement generator that generates information related to head movement in the m-th (natural number from 1 to n) video using the information related to the head coordinates generated from the at least one artificial neural network model;
an eye movement generator that generates information related to eye movement in the m-th video using the information related to the coordinates of the pupil center or the eye phase changes generated from the at least one artificial neural network model;
a speed information generator that generates information related to head and eye movement speeds in the m-th video using the information related to the head movement and the eye movement; and
a balance function status information generator that generates information on a balance function status of the subject using the information related to the head and eye movement speeds.

13. The balance function management system according to claim 12, wherein the speed information generator generates the information related to the head movement speed and the eye movement speed within a preset time based on a time when the head movement becomes greater than or equal to a preset threshold value.

14. The balance function management system according to claim 12, wherein the balance function status information generator calculates a gain using a time value at which the head movement speed is maximum and a time value at which the eye movement speed is maximum.

15. The balance function management system according to claim 12, wherein, when n is greater than or equal to 2,
the head movement generator further generates reference head movement information by calculating statistical values of the information related to the head coordinates according to the m-th video, and
the eye movement generator further generates reference eye movement information by calculating statistical values of the information related to the coordinates of the pupil center and the eye phase changes according to the m-th video.

16. The balance function management system according to claim 1, wherein the at least one processor comprising:
a head movement generator that generates the information related to head movement in the m-th (natural number from 1 to n) using the information related to the head coordinates generated from the at least one artificial neural network model;
an eye movement generator that generates the eye movement information in the m-th video using the information related to the coordinates of a pupil center or the eye phase changes acquired from the at least one artificial neural network model;
a target output generator that outputs a virtual target to a display device;
a head direction provider that provides direction information on the head movement to the subject; and
a feedback provider that provides feedback according to the head movement and the eye movement of the subject.

17. A method of generating information on a balance function status in a balance function management system including at least one processor and a memory that stores instructions executable by the processor and stores at least one artificial neural network model executed on a computing device, the method comprising:
acquiring, by the at least one processor, information related to head coordinates, coordinates of a pupil center, and eye phase changes of a subject according to an order of frame images of an m-th (natural number from 1 to n) video by allowing the at least one processor to input frame images of n videos of the subject captured by n (natural number) cameras to at least one artificial neural network model; and
generating, by the at least one processor, head movement information and eye movement information using the information acquired from the at least one artificial neural network model, calculating a head movement speed and an eye movement speed, and generating information related to a balance function status using information related to the head movement speed and the eye movement speed.

18. A balance function rehabilitation method in a balance function management system including at least one processor and a memory that stores instructions executable by the processor and stores at least one artificial neural network model executed on a computing device, the balance function rehabilitation method comprising:
acquiring, by the at least one processor, information related to head coordinates, coordinates of a pupil center, and eye phase changes of a subject according to an order of frames of an m-th (natural number from 1 to n) video by allowing the at least one processor to input frame images of n videos of the subject captured by n (natural number) cameras to the at least one artificial neural network model; and
generating, by the at least one processor, head movement information and eye movement information using the information acquired from the at least one artificial neural network model and performing a balance function rehabilitation program by head and eye movements of the subject.

19. A computer program written to perform the method of generating information on a balance function status according to claim 17 on a computer and recorded on a computer-readable recording medium.

20. A computer program written to perform the balance function rehabilitation method according to claim 18 on a computer and recorded on a computer-readable recording medium.
